(19) United States
(12) Patent Application Publication
(10) Pub. No.: US 2026/0045005 A1
(43) Pub. Date: Feb. 12, 2026

(54) SYNTHETIC DATA GENERATION FOR MODALITY-AGNOSTIC ZERO-SHOT FOUNDATION MODEL FOR MEDICAL IMAGES

(71) Applicant: GE Precision Healthcare LLC, Waukesha, WI (US)

(72) Inventors: Satrajit Chakrabarty, San Ramon, CA (US); Sourya Sengupta, Urbana, IL (US); Keerthi Sravan Ravi, Pleasanton, CA (US); Ravi Soni, Livermore, CA (US); Gopal Biligeri Avinash, Concord, CA (US)

(21) Appl. No.: 19/291,155

(22) Filed: Aug. 5, 2025

Related U.S. Application Data
(60) Provisional application No. 63/680,300, filed on Aug. 7, 2024.
(60) Provisional application No. 63/680,516, filed on Aug. 7, 2024.

(51) Int. Cl. G06T 11/20; G06T 5/30; G06V 10/762; G16H 30/20; G16H 30/40

(52) U.S. Cl. CPC G06T 11/203; G06T 5/30; G06V 10/762; G16H 30/20; G16H 30/40

(57) ABSTRACT

One or more systems, devices, computer program products and/or computer-implemented methods of use provided herein relate to assessing certainty of artificial intelligence models used for detection or segmentation of pathologies. Accordingly, a system can comprise a memory that can store computer executable components. The system can further comprise a processor that can execute at least one of the computer executable components. The computer executable components can comprise a synthetic data generation component that generates biologically-inspired synthetic data that approximates a task-specific data manifold of a medical image from a radiomic features perspective; an artificial intelligence component that uses an artificial intelligence model to learn relevant representations of the synthetic data for an at least one image task; and a training component that utilizes the relevant representations and the artificial intelligence model to generate a task-specific model for the at least one image analysis task.

### CROSS REFERENCE TO RELATED APPLICATIONS

This application claims priority to and the benefit of U.S. Provisional Patent Application No. 63/680,516, filed Aug. 7, 2024, and U.S. Provisional Patent Application No. 63/680,300, filed Aug. 7, 2024, the entire contents of each of which are incorporated by reference herein in their entirety.

### TECHNICAL FIELD

The subject disclosure relates generally to a synthetic data generation method that captures key data characteristics such as contrast, noise, and captures image features such as, but not limited to, contrast, noise, texture, and boundaries between objects that typically characterize data commonly observed in medical imaging.

### BACKGROUND

In recent years, foundation models have surged in popularity within the field of artificial intelligence. These models, typically implemented as large-scale deep neural networks, are trained on massive and diverse datasets and demonstrate impressive generalization capabilities across a wide variety of downstream tasks. Unlike conventional task-specific models, foundation models can often perform well on new tasks in a zero-shot or few-shot manner, having learned broad and transferable representations during pretraining. This makes them especially attractive for applications where labeled data is limited or unavailable.

However, despite their versatility, foundation models do not always generalize well to specialized domains such as medical imaging. Medical images, including those from computed tomography (CT), magnetic resonance imaging (MRI), ultrasound, and other modalities, differ from natural images in fundamental ways. They often exhibit unique noise characteristics, modality-specific contrast profiles, non-uniform textures, and complex anatomical structures with ambiguous boundaries. These differences can cause foundation models trained primarily on natural image datasets to perform poorly when applied to medical imaging tasks.

The Segment Anything Model (SAM), for example, is a foundation model designed for promptable image segmentation. It has shown exceptional zero-shot performance on natural images, largely due to its exposure to an extensive training corpus of annotated masks and its prompting capabilities. Despite this, SAM has demonstrated limited effectiveness in the medical domain. Its segmentation outputs often fail to capture the subtle distinctions between anatomical structures or are confounded by the relatively low contrast and high variability present in medical data. These issues can result in over-segmentation, under-segmentation, or failure to identify target regions entirely.

One possible solution to this domain mismatch is to fine-tune foundation models on medical datasets. However, obtaining large-scale, high-quality annotated medical image datasets is a significant challenge. Annotations typically require expert knowledge from trained radiologists or other medical professionals, making the process time-consuming, labor-intensive, and expensive. Moreover, patient privacy regulations, data heterogeneity across institutions, and variability in acquisition protocols further complicate the creation of comprehensive medical datasets. As a result, efforts to adapt foundation models like SAM to the medical domain must contend with both technical and practical obstacles related to data availability and domain adaptation.

Accordingly, systems or techniques that can address one or more of these technical problems can be desirable.

### SUMMARY

The following presents a summary to provide a basic understanding of one or more embodiments. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatus or computer program products can facilitate automatic product support via generative artificial intelligence and customer interactions.

At a high-level, one or more non-limiting embodiments relate to leveraging expert domain expertise to facilitate synthetic data modeling to finetune or train any foundation model without requirement of real data, enabling superior zero-shot performance on image segmentation. Another non-limiting embodiment relates to a systematic medical imaging specific synthetic data modeling approach that was used to finetune SAM without requirement of real data, enabling superior zero-shot performance on medical image segmentation.

According to one or more embodiments, a system is provided. The system can comprise a non-transitory computer-readable memory that can store computer-executable components. The system can further comprise a processor that can execute the computer-executable components. The computer-executable components can include a synthetic data generation component that can generate biologically-inspired synthetic data that closely approximates a task-specific data manifold of a medical image from a radiomic features perspective. The computer-executable components can further include an artificial intelligence component that can an artificial intelligence model to learn relevant representations of the synthetic data to generate task-specific outputs for an at least one image task. The computer-executable components can additionally include a training component that can utilize the synthetic data and the artificial intelligence model to generate a task-specific model for the at least one image analysis task.

According to one or more embodiments, a computer-implemented method is provided. In various embodiments, the computer-implemented method can comprise generating, by a system operatively coupled to a processor, biologically-inspired synthetic data that closely approximates a task-specific data manifold of a medical image from a radiomic features perspective by analytically compositing foreground and background components. In various aspects, the method can comprise using an artificial intelligence model to learn relevant representations of the synthetic data to generate task-specific outputs for an at least one image task. The method can further comprise utilizing the relevant representations of the synthetic data and the artificial intelligence model to generate a task-specific model for the at least one image analysis task. In some embodiments, the method can additionally include generating a multiclass label map comprising multiple clusters, selecting a subset of the clusters, and performing a randomly selected number of erosion operations on the selected clusters to define thin boundaries between adjacent anatomical structures.

According to one or more embodiments, a computer program product for facilitating training of an image analysis model is provided. In various embodiments, the computer program product can comprise a non-transitory computer-readable memory having program instructions embodied therewith. The program instructions can be executable by a processor to cause the processor to generate biologically-inspired synthetic data that closely approximates a task-specific data manifold of a medical image from a radiomic features perspective. The program instructions can further be executable to cause the processor to use an artificial intelligence model to encode features of the synthetic data for at least one image analysis task. Additionally, the program instructions can be executable to cause the processor to utilize the features of the synthetic data and the artificial intelligence model to generate a task-specific model for the at least one image analysis task.

### DESCRIPTION OF THE DRAWINGS

FIG. 1 illustrates a block diagram of an example, non-limiting system that facilitates automatic product support via generative artificial intelligence and customer interactions in accordance with one or more embodiments described herein.

FIG. 2 illustrates a block diagram of an example, non-limiting system that facilitates automatic product support via generative artificial intelligence and customer interactions in accordance with one or more embodiments described herein.

FIG. 3 illustrates a flow diagram of an example, non-limiting computer-implemented method that facilitates automatic product support via generative artificial intelligence and customer interactions in accordance with one or more embodiments described herein.

FIG. 4 illustrates a flow diagram of an example, non-limiting computer-implemented method that facilitates automatic product support via generative artificial intelligence and customer interactions in accordance with one or more embodiments described herein.

FIG. 5 illustrates image segmentation in accordance with one or more embodiments described herein.

FIG. 6 illustrates example variations of training images with respect to a target in accordance with one or more embodiments described herein.

FIG. 7 illustrates a single prompt & single object; multiple prompts & single object; and multiple prompts & multiple objects in accordance with one or more embodiments described herein.

FIG. 8 illustrates a framework for various embodiments in accordance with one or more embodiments described herein.

FIG. 9 illustrates a system-level overview of how synthetic data with medical realism can be used to train a foundation model that generalizes across a range of clinical imaging contexts in accordance with one or more embodiments described herein.

FIG. 10 illustrates a diagram of a synthetic image generation and annotation pipeline in accordance with one or more embodiments described herein.

FIG. 11 illustrates an example synthetic shape generation process that can employ Bézier curves to produce anatomically inspired geometries and spatial configurations in accordance with one or more embodiments described herein.

FIG. 12 illustrates a synthetic image generation workflow that can combine background textures with label map overlays to produce realistic training samples in accordance with one or more embodiments described herein.

FIG. 13 illustrates the effect of a boundary-aware shape generation module on a multiclass segmentation mask in accordance with one or more embodiments described herein.

FIG. 14 illustrates an example workflow for generating synthetic medical image textures using a boundary-aware binarized mask and random background canvas in accordance with one or more embodiments described herein.

FIG. 15 illustrates depicts examples of synthetic data generated using a shape-aware module and a boundary-aware module in accordance with one or more embodiments described herein.

FIG. 16 illustrates a summary of evaluation datasets used to validate performance across diverse clinical imaging scenarios in accordance with one or more embodiments described herein.

FIG. 17 illustrates a quantitative comparison of segmentation performance across multiple anatomical structures, imaging modalities, and model configurations in accordance with one or more embodiments described herein.

FIG. 18 illustrates a visual comparison of segmentation performance across multiple models using representative clinical imaging samples for different anatomical structures and modalities in accordance with one or more embodiments described herein.

FIG. 19 illustrates a block diagram of an example, non-limiting operating environment in which one or more embodiments described herein can be facilitated.

FIG. 20 illustrates an example networking environment operable to execute various implementations described herein.

### DETAILED DESCRIPTION

The following detailed description is merely illustrative and is not intended to limit embodiments or application/uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

Foundation models are large-scale models trained on extensive datasets, which demonstrate excellent zero-shot generalization capabilities as a result of a comprehensive training process. Meta's® recently released Segment Anything Model (SAM), trained on a massive corpus of 11 million images and 1 billion masks, demonstrates superior performance in promptable image segmentation. SAM offers remarkable zero-shot segmentation capabilities on unseen data, which can be attributed to the immense variety of data that it was exposed to during the training phase. Accurate image segmentation is critical in computer-aided diagnosis for isolating anatomical objects of interest. SAM's impressive zero-shot performance and prompting ability have sparked interest in exploring its application to medical image segmentation. However, obtaining task-specific annotations is expensive, time-consuming, and labor-intensive, as it requires the expertise of highly skilled clinicians. Additionally, task-specific deep learning models often suffer performance degradation on real-world data due to subtle differences in data characteristics, resulting in the data being out-of-domain for the trained model. This issue is not always solvable through robust data preprocessing techniques. Furthermore, training task-specific models can quickly become cumbersome. Zero-shot models with robust generalization to unseen data can potentially mitigate these challenges. Additionally, a zero-shot model that supports prompting, such as SAM, can enable interactive image segmentation, allowing users to specify objects of interest and correct errors. However, SAM has shown poor performance in medical image segmentation tasks. This limitation can be attributed to the fact that medical imaging data is out-of-domain for SAM, as it was trained exclusively on natural images, which differ significantly from medical images in terms of contrast, texture, and noise. For instance, natural images typically have distinct boundaries between objects and the background, which is not always the case in medical imaging modalities like CT and label-free microscopy, where organ boundaries are often fuzzy. Consequently, SAM struggles to differentiate between organ boundaries and may segment the entire image as a single object.

A zero-shot promptable model like SAM is disclosed that can be adapted for medical image segmentation by fine-tuning it on synthetic data that closely approximates a data manifold of medical images. A synthetic data generation method captures key data characteristics such as contrast, noise, and textures commonly observed in medical imaging. The model encoders are initialized with SAM-pretrained weights and a complete model is trained on this synthetic dataset. Extensive experiments were conducted to evaluate its performance on 12 different anatomical structures from three imaging modalities across eight publicly available datasets. Using only a single positive prompt in all the experiments, it was observed that the SynthFM approach consistently outperforms the original SAM across all datasets evaluated. This innovation is a synthetic data generation method for fine-tuning a foundational model to enable generalization to real-world medical imaging data.

### Data Generation Strategy

SynthFM captures the key aspects of medical imaging, which are different from natural images, such as simulating diverse anatomical shapes, varying contrasts and textures, and generating structures in close proximity to adjacent anatomical features.

### Shape-Aware Module

Medical images exhibit diverse anatomical shapes and structures, varying across organs and patients. To capture this variability, a B′ezier curve-based object generation approach was adapted. A B′ezier curve is defined by n+1 control points P₀, P₁, . . . , Pₙ. Its curve B (t) for t ∈ [0, 1] is expressed as

$$B ( t ) = {\sum }_{i = 0}^{n} ( \begin{matrix}n \\ i\end{matrix} ) {( 1 - t )}^{n - i} {t}^{i} {P}_{i} , where ( \begin{matrix}n \\ i\end{matrix} )$$

is the binomial coefficient. The terms (1−t)ⁿ⁻ⁱtⁱ denote Bernstein polynomials, which ensure that the curve starts at P₀ when 1=0 and ends at Pₙ when t=1. The control points heavily influence the curve's shape, pulling it toward these points without necessarily passing through them (except for the endpoints P₀ and Pₙ). By randomly sampling different number of control points, a diverse set of shapes and structures were created in each case.

### Texture and Noise Library.

The unique texture and contras characteristics of medical images set them apart from natural images, where object-background contrast is typically high. Medical images often feature subtle contrast variations, making structure differentiation more challenging. In the disclosed method, objects generated by the shape-aware module were superimposed on a background that was either a black canvas or a black canvas with a phantom (simple circular shape) overlaid, mimicking common imaging modalities, where objects appear against uniform backgrounds.

To simulate the range of noise and texture variations found in real medical images, a noise library was developed, including noise models such as additive Gaussian noise, multiplicative Poisson noise, Perlin noise, speckle noise, and Rician noise. These noise patterns were generated on-the-fly and randomly applied to the shapes during training. Additionally, Gaussian blurring was introduced to replicate the blurriness often seen around organ edges in medical images.

To simulate varying contrast levels between anatomical structures and backgrounds, the contrast was modulated using the heuristic function (1−p)·(m·r), where p is the phantom or background intensity, m indexes each shape (from 1 to the total number of shapes), and r is a randomly sampled variable from the interval [−0.2, 0.2]. The intensity p was drawn from a uniform distribution p˜U(0, 1) for each case, ensuring a wide range of intensity backgrounds. This formulation generated diverse intensity variations in synthetic images by adjusting contrast between structures and background.

### Boundary-Aware Module.

The shape-aware module captured individual shape complexities but generated shapes independently, resulting in disconnected structures. In medical images, however, structures often share boundaries with minimal contrast differences, complicating segmentation. To address this, the boundary-aware module was developed to generate adjacent structures with shared boundaries. SynthMorph was leveraged to generate a multiclass label map with 10-15 labeled clusters. A subset of clusters was then randomly selected and eroded for a randomly chosen number of iterations to introduce boundaries between adjacent structures. After erosion, all clusters were assigned the foreground label and the boundary created from erosion was assigned the background label, resulting in a simplified binary mask.

### Contrast and Texture.

The training image was generated based on the binary mask by assigning intensity values from a randomly generated canvas with controlled variations. The canvas was populated with values from a randomly chosen range [limit1, limit2], limit1 ∈ [0.1, 0.9] and limit2 being either slightly below or above limit1, and within [0, 1]. To introduce variability, a small proportion of canvas values was perturbed outside this range, scattered randomly across the entire interval [0, 1]. The canvas was then combined with the binary mask: for foreground areas, the values from the random canvas were directly applied. For background areas, the canvas values were averaged and a small random perturbation was added, producing a slight positive or negative shift. This ensured nuanced variations across the image, enhancing the complexity and realism of the synthetic data.

### Model Architecture and Training Details.

The SAM architecture was utilized in experiments. Dice loss was used for training, with encoders initialized from the original SAM weights while the decoder was trained from scratch. Each epoch included 10,000 images of size 1024×1024, with a batch size of 1, and data was generated on-the-fly during training. The model was trained for 100 epochs with a learning rate of 1×10 4 using cosine decay, and the model with the best validation performance was selected.

### Prompt Strategy

SynthFM's performance was evaluated using four positive-negative prompt configurations: (1, 0), (3, 0), (1, 2), and (3, 2), where the two values represent the number of positive and negative clicks respectively. For positive prompts, the first prompt was placed near the target structure's centroid, with additional prompts randomly positioned within the target structure. For negative prompts, the prompts were selected from a dilated region around the target structure-ensuring the first prompt was maximally distant from the centroid, with subsequent negative prompts spaced apart from each other.

Various embodiments described herein can address one or more of the above-described technical problems. One or more embodiments described herein can include systems, computer-implemented methods, apparatus, or computer program products that can facilitate automatic product support via generative artificial intelligence and customer interactions. In particular, the inventors of various embodiments described herein realized that foundation models trained solely on natural images suffer from severe performance degradation when applied to medical image segmentation tasks due to domain mismatch. More particularly, the inventors realized that generating synthetic medical imaging data that accurately reflects the structural and statistical characteristics of real medical images could enable effective adaptation of foundation models, such as SAM, to medical tasks-without requiring any manually labeled medical images.

Accordingly, various embodiments described herein can be considered as improving the field of medical image segmentation by enabling foundation model adaptation without real data. The ability to simulate both the anatomical complexity and image-level noise characteristics of medical images can ensure that a model exposed only to synthetic data can still generalize effectively to real-world imaging modalities, allowing for scalable and low-cost deployment in clinical or diagnostic workflows.

Accordingly, systems described herein can enhance the utility of zero-shot segmentation models by bridging the domain gap between natural and medical images through analytical synthetic data generation, including both shape-aware and boundary-aware components combined with modality-specific texture modeling.

Various embodiments described herein can be considered as a computerized tool that can facilitate training of artificial intelligence models for image analysis tasks using synthetic data generation. Such tools can comprise a synthetic data generation component, an artificial intelligence component configured to encode features from synthetic data, a training component for generating a task-specific model, and optionally include shape-aware, boundary-aware, and contrast and texture modules for enhancing the biological and radiomic realism of synthetic data.

In various embodiments, the synthetic data generation component can generate biologically-inspired synthetic data that closely approximates a task-specific data manifold of a medical image from a radiomic features perspective. The synthetic data can emulate structural, statistical, and visual characteristics of imaging modalities such as computed tomography (CT), magnetic resonance imaging (MRI), ultrasound, and label-free microscopy. The synthetic data generation component can include a shape-aware module, a boundary-aware module, and a contrast and texture modeling module. These modules can collaboratively model anatomical structure, adjacency and interaction, and texture-related realism, enabling generation of synthetic training images that reflect realistic biological variation, imaging artifacts, and diagnostic challenges.

The shape-aware module of the synthetic data generation component can employ Bézier curve-based object generation to construct diverse anatomical structures. The module can randomly select a number of control points for each Bézier curve to vary the shape complexity. The generated shapes can be blurred using Gaussian filters with randomly selected sigma values to simulate soft tissue boundaries commonly found in medical images. The module can superimpose the generated shapes onto background canvases that are either uniform or include simple geometric phantoms. To simulate contrast differences between anatomical structures and background, the component can adjust intensity values according to a contrast modulation function of the form (1−p)×(m×r), where p is a phantom or background intensity value sampled from a uniform distribution, m is the index of the shape, and r is a randomly sampled variability parameter. This configuration enables the generation of a wide range of anatomical intensities and structural contrasts.

The synthetic data generation component can further include a noise and texture augmentation pipeline configured to apply realistic imaging noise patterns. The component can access a noise library comprising additive Gaussian noise, multiplicative Poisson noise, Rician noise, Perlin noise, and speckle noise. During training, one or more of these noise types can be applied on-the-fly to each generated image to emulate texture and noise conditions encountered in real imaging modalities. Additionally, the boundary-aware module can generate adjacent anatomical structures with thin or ambiguous boundaries. This module can generate multiclass label maps with 10-15 clusters, select a subset of the clusters, and apply erosion operations with randomly selected iteration counts to produce thin boundary regions. The clusters can then be collapsed into binary masks distinguishing foreground from background. Foreground regions can be assigned pixel values from randomized intensity canvases, and background regions can be generated by averaging canvas values and introducing small perturbations. The system can support not only on-the-fly (real-time) synthetic data generation but also offline generation and persistent storage of synthetic datasets. The system can be configured to save each generated synthetic image along with comprehensive metadata describing all parameters, configurations, and operations applied during the data generation process. This metadata can include, but is not limited to, shape control points, noise types and intensity, canvas configurations, contrast modulation parameters, and prompt placement strategies. By retaining both the data and associated metadata, the system can enable reproducibility and repeatability of synthetic dataset creation, allowing datasets to be regenerated or verified by external systems. This capability can further facilitate data sharing, standardized benchmarking, and compliance with regulatory or institutional reproducibility requirements. The system can thereby support dynamic, in-memory data generation during training as well as pre-generated datasets that are saved, versioned, and distributed for external consumption.

In various embodiments, an artificial intelligence component can use an artificial intelligence model to encode features of biologically-inspired synthetic data for at least one image analysis task. The artificial intelligence component can use an artificial intelligence model to learn relevant representations of the synthetic data to generate task-specific outputs for an at least one image task. The artificial intelligence model can comprise a general-purpose or pre-trained architecture (e.g., a convolutional neural network, vision transformer, or foundation model) that can be adapted to extract meaningful visual features from biologically-inspired synthetic images. The artificial intelligence component can process the synthetic data to generate latent representations that capture semantic, structural, and radiomic characteristics aligned with the intended task, such as segmentation, classification, or detection. These learned representations can serve as the internal basis for producing task-specific outputs, including but not limited to anatomical masks, region labels, or confidence maps. By leveraging synthetic data tailored to the radiomic properties and spatial configurations of specific imaging tasks, the artificial intelligence model can learn to generalize across diverse imaging modalities without requiring real-world clinical annotations. This configuration can enable the artificial intelligence component to bridge domain-specific priors embedded in synthetic data with the flexible pattern recognition capabilities of a general-purpose model, facilitating downstream performance in medical image analysis.

The artificial intelligence component can process synthetic medical images generated by a synthetic data generation pipeline and convert them into high-dimensional feature representations that capture structural, contextual, and radiomic characteristics relevant to the task at hand. The artificial intelligence model can include encoder architectures derived from general-purpose models such as convolutional neural networks, vision transformers, or foundation models trained on large image corpora. By applying the artificial intelligence model to the synthetic data, the component can produce task-relevant embeddings that preserve fine-grained spatial information, texture details, and semantic cues critical for downstream applications such as segmentation, classification, or detection. These encoded features can serve as input to a training pipeline that further refines or adapts the model to perform the target image analysis task using only synthetic training signals, thus avoiding reliance on manually annotated clinical data.

In various embodiments, the artificial intelligence component can encode features of the synthetic data using a general-purpose artificial intelligence model. This model can include encoder structures adapted from foundation models such as the Segment Anything Model (SAM) or other deep neural networks. The encoders can generate high-dimensional embeddings from the synthetic inputs, preserving spatial and semantic information relevant to downstream tasks. The AI component can be used in conjunction with decoders or other architectural modules to form end-to-end pipelines for training task-specific models.

In various embodiments, the artificial intelligence component can use an artificial intelligence model to process the synthetic data and generate intermediate feature representations that facilitate downstream model training. These feature representations can be latent vectors or embeddings that capture structural, semantic, and radiomic characteristics of the synthetic data, including attributes such as edge sharpness, tissue intensity gradients, shape irregularity, and spatial adjacency. The artificial intelligence model can comprise a general-purpose encoder, such as a convolutional neural network (CNN) or a vision transformer (ViT), optionally pre-trained on large-scale image datasets. Upon receiving synthetic medical images, the encoder can transform these inputs into a structured feature space that reflects both low-level texture patterns and high-level anatomical structures. These learned representations can serve as input to a task-specific training component that fine-tunes decoder layers or specialized heads to perform segmentation, classification, or other image analysis tasks. In some implementations, the encoder can remain fixed while only the downstream task-specific layers are trained, whereas in others, the encoder can be jointly optimized to refine its feature encoding. The use of feature representations rather than raw input images allows for more efficient and generalizable model training, bridging the synthetic-to-real domain gap while preserving diagnostic and morphological relevance.

In various embodiments, the encoders can be initialized with pre-trained weights for a pre-trained model for at least one task. The encoders can be initialized with pre-trained weights for a pre-trained model configured to perform at least one image segmentation task. The encoders can include components from a foundation model architecture, such as the image encoder of the Segment Anything Model (SAM). The pre-trained weights can be derived from training on large-scale natural image datasets comprising diverse objects and segmentation masks. By initializing the encoders with these pre-trained weights, the system can leverage learned representations that generalize well to image features such as edges, textures, and object boundaries, even in domains different from those present in the original training set.

The encoders can be integrated into a segmentation architecture in combination with a decoder and, optionally, a prompt encoder. During training, the system can retain the pre-trained encoder weights in a frozen or semi-frozen state, allowing only the decoder- or selected trainable layers—to be updated based on the synthetic medical training data. This training strategy can preserve the broad representational capacity of the foundation model while adapting downstream segmentation capabilities to medical imaging tasks. The encoder can process synthetic input images at high resolution and generate feature embeddings that are consumed by the decoder to produce segmentation masks aligned with anatomical structures present in the synthetic data.

The set of encoders (e.g., the artificial intelligence component) can support on-the-fly training workflows, where synthetic images are dynamically generated and immediately passed through the encoder during each training iteration. This configuration can enable efficient model convergence while avoiding the need to precompute or store large training datasets. The use of pre-trained encoders initialized with general-purpose vision weights can reduce the amount of synthetic data required for effective training and can improve the ability of the resulting model to generalize to real-world medical imaging inputs.

In various embodiments, the training component can utilize the synthetic data and the artificial intelligence model to generate a task-specific model for the at least one image analysis task. The training component can receive biologically-inspired synthetic images (e.g., generated to approximate a task-specific data manifold from a radiomic features perspective) and apply them as inputs to the artificial intelligence model to fine-tune or adapt the model toward a targeted clinical outcome. The training component can update selected parameters of the artificial intelligence model (e.g., decoder layers, task-specific heads) based on supervision signals derived from synthetic labels or masks associated with each synthetic image. The training process can employ optimization routines such as stochastic gradient descent or Adam, using loss functions tailored to the intended image analysis task (e.g., Dice loss for segmentation or cross-entropy for classification). By systematically exposing the artificial intelligence model to a high-variability synthetic training set, the training component can enable the emergence of a specialized model that performs reliably in the context of a specific imaging task. This approach can eliminate the need for manually labeled real-world medical data, while promoting generalization to diverse anatomical structures, imaging artifacts, and modality-specific characteristics.

In various embodiments, the training component can utilize the generated synthetic data and set of encoders to generate the pre-trained model for the at least one task. The training component can utilize the generated synthetic data and the set of encoders to generate a trained model for at least one image segmentation task. The training component can receive synthetic medical images along with corresponding masks derived from the synthetic generation process, and can use these as supervision signals to optimize the parameters of a segmentation model. The component can implement a supervised learning pipeline wherein the encoders are initialized with pre-trained weights, and the decoder is trained from scratch or fine-tuned to learn segmentation mappings specific to medical imaging structures.

In various embodiments, the training component can utilize the synthetic data and the encoded features to generate a task-specific model for at least one image analysis task. This component can receive as input both the biologically-inspired synthetic medical images and the feature embeddings produced by an artificial intelligence component, and can optimize the parameters of a downstream model to perform a defined task such as image segmentation, classification, or localization. The training component can implement a supervised or semi-supervised learning pipeline using labels or masks derived from the synthetic generation process as ground truth. By leveraging the encoded feature representations, the component can train lightweight task-specific decoders or adapt modular components of a larger architecture, such as fine-tuning prompt interpreters or segmentation heads. The training process can include loss functions tailored to the image analysis task-such as Dice loss, cross-entropy, or contrastive loss- and can support various learning rate schedules, optimizer strategies, and regularization techniques. The resulting model can generalize effectively to real clinical imaging data without having been exposed to manually annotated examples, thereby enabling efficient and scalable deployment across a range of medical image analysis applications.

The training component can employ a loss function such as Dice loss to measure the similarity between predicted segmentation masks and ground truth synthetic masks. Optimization can be performed using gradient descent-based algorithms, with learning rate schedules such as cosine decay applied over a defined number of training epochs. Each training epoch can consist of a specified number of synthetic image-mask pairs, and the synthetic data can be generated dynamically to ensure high variability and prevent overfitting. The training component can evaluate model performance on a validation subset and select the best-performing model checkpoint based on segmentation accuracy.

The training component can support various training configurations, including freezing the encoder layers initialized with pre-trained weights while training only the decoder, or selectively fine-tuning encoder layers as needed. The component can operate in a low-resource setting with a batch size of one and high-resolution input images, such as 1024×1024 pixels. By leveraging dynamically generated synthetic data and pre-trained encoder representations, the training component can enable efficient domain adaptation of general-purpose foundation models to specialized medical segmentation tasks-without requiring any manually annotated real medical data.

The shape aware synthetic tool component can employ Bézier curve-based object generation to construct a diverse set of synthetic anatomical structures used for training the artificial intelligence model. Bézier curves provide a mathematically controlled way to generate smooth and continuous shapes based on a series of control points. The component can randomly select the number and placement of Bézier control points for each instance, allowing for extensive variability in the geometry and complexity of the generated shapes. The resulting curves can simulate the irregular contours and orientations of biological organs, vessels, and tissues, making the synthetic shapes more anatomically realistic. The generated shapes can be instantiated as foreground structures against uniform or phantom-containing backgrounds, forming segmentation masks paired with their corresponding synthetic image inputs. By generating a large number of distinct shapes with controlled randomness, the system can ensure that the model is exposed to a wide distribution of structural patterns during training. This diversity can help the model learn generalizable shape priors and mitigate overfitting. The shape aware synthetic tool component can also allow for additional post-processing such as resizing, rotation, translation, and affine transformations to further increase spatial variability. In some embodiments, the shape generation process can be seeded or conditioned to reflect known organ geometries using parameterized control point patterns. For example, the tool can incorporate shape templates derived from statistical models of organ morphology and add controlled perturbations to generate varied yet realistic synthetic instances. This configuration allows the system to simulate population-level anatomical variability, including variations due to patient age, sex, or pathology. The output of the shape aware module can be stored as binary masks and paired with synthetic textures and intensities generated by other components for full-scene medical image simulation.

The contrast and texture tool component can enhance synthetic image realism by simulating the grayscale intensity dynamics, blurriness, and noise characteristics common to medical imaging modalities. It can apply multiple image augmentation operations such as Gaussian noise, Gaussian blurring, and phantom intensity overlays to replicate modality-specific acquisition artifacts. Gaussian noise can be added with varying standard deviation levels to emulate low-signal environments, sensor variability, or electronic noise in imaging systems such as ultrasound or low-dose CT. Gaussian blurring can be used to degrade the sharpness of anatomical edges, simulating phenomena such as motion blur, low spatial resolution, or partial volume effects. The tool can vary the blurring kernel size and sigma values to apply different levels of edge softness to each image, reinforcing the model's ability to localize structures under uncertain conditions. In some instances, the background canvas can be overlaid with circular phantom shapes or smooth gradients to represent the homogeneous appearance of soft tissue regions commonly found in CT and MR scans. These phantoms can serve as background anchors that enhance the perceived realism of spatial context within the image. The component can also apply controlled intensity modulation across anatomical structures using a formula such as (1−p)×(m×r), where p is a phantom or background intensity value sampled from a uniform distribution, m represents the shape index, and r is a random number from the interval [−0.2, 0.2]. This modulation introduces contrast differences across shapes and between foreground and background, replicating the subtle grayscale variations characteristic of many medical modalities. By dynamically varying these parameters, the contrast and texture tool component can produce a synthetic dataset with high visual diversity and realistic appearance, enabling robust model training without relying on real-world annotated medical data.

The boundary-aware synthetic tool component can simulate anatomical structures that are spatially adjacent and difficult to differentiate due to thin or low-contrast boundaries. This scenario frequently occurs in medical imaging, where organs or tissues of similar intensity are positioned in close proximity. The component can use a structured approach to generate such conditions, leveraging tools like SynthMorph to create initial label maps with multiple distinct regions or clusters. These label maps can then be manipulated to simulate complex adjacency and interaction between anatomical structures. The component can randomly select a subset of clusters within a generated label map and apply morphological erosion operations to reduce their spatial extent. The number of erosion iterations can be sampled from a weighted random distribution, favoring lower iteration counts to produce very narrow boundaries. This technique can mimic the appearance of tightly packed anatomical regions, such as abdominal organs, where boundaries are hard to distinguish. After erosion, a binary segmentation mask can be created by labeling all non-background regions as foreground, effectively simplifying the map while preserving the complexity of structure adjacency. The boundary-aware synthetic tool can thereby generate training data that presents meaningful segmentation challenges to the model. These thin-boundary scenarios can force the model to learn finer spatial distinctions and better understand context across adjacent regions. In some configurations, the component can introduce partial overlap or intensity similarity across structures, further increasing the difficulty of the segmentation task. This process can significantly enhance the generalizability of the model by exposing it to edge cases that often lead to failure in real-world inference settings.

The synthetic data generation component can be configured to produce synthetic medical images that accurately emulate the visual appearance of real-world scans, including properties such as grayscale distribution, noise level, edge sharpness, and anatomical density. This is achieved by coordinating the operations of the shape aware, contrast and texture, and boundary-aware modules in a pipeline. Each synthetic image can be constructed from scratch by generating synthetic structures, compositing them against a textured or phantom background, and applying contrast modulation and noise patterns in real time. The shape aware synthetic tool component can specifically control the shape complexity of the generated anatomical structures by varying the number of Bézier curve control points. Increasing the number of control points can result in more intricate and irregular shapes, while fewer control points can yield simpler, smoother geometries. This mechanism enables the system to represent both large, well-defined organs (e.g., liver, kidney) and small, irregular structures (e.g., tumors, ducts), giving the trained model exposure to a wide spectrum of segmentation targets. The variability in shape complexity can be essential for training models that generalize across clinical use cases. For example, a model exposed only to regular, easily segmented shapes may fail to correctly identify or delineate pathologies that distort organ boundaries. By generating a mix of simple and complex shapes within the synthetic dataset, the system can simulate a broader distribution of clinical imaging appearances and improve the adaptability of the trained model across patient populations and disease states.

The contrast and texture tool component can include a noise library configured to introduce multiple types of noise to the synthetic training images. This noise library can be used to simulate real-world acquisition noise present in different imaging modalities. The available noise types can include Poisson noise, which models photon shot noise in low-light conditions; Rician noise, which arises in magnitude-reconstructed MR images; speckle noise, typical in ultrasound imaging; and Perlin noise, which can add organic-looking texture variations. These noise types can be applied alone or in combination to foreground, background, or the entire image canvas. During training, the system can randomly select one or more noise models and apply them with varying strength and spatial distribution. This randomization can occur on a per-image basis or at the patch level, ensuring that the synthetic data distribution remains broad and non-redundant. The ability to inject multiple types of noise across spatial and temporal dimensions can help the model become robust to noise-induced segmentation artifacts and generalize well to real-world imaging conditions. The noise library can be tightly integrated with other synthetic data generation modules to ensure that noise is applied after contrast modulation and structural rendering, thereby preserving the semantic integrity of the synthetic annotations. The component can also be configured to maintain consistent noise levels across multiple training epochs or introduce gradual noise escalation strategies, which can support curriculum-based learning workflows for more stable model convergence.

The boundary-aware synthetic tool component can include an erosion module that creates narrow boundaries between adjacent synthetic structures through morphological processing. The module can begin by generating a multiclass label map with multiple anatomical clusters. A subset of these clusters can be selected for boundary simulation, and an erosion operation can be applied to reduce the size of each selected cluster. The erosion kernel size and the number of iterations can be randomly selected, allowing for fine-grained control over the resulting boundary thickness. Once the erosion process is complete, the clusters can be merged into a binary mask where foreground areas include all eroded clusters, and the space between them is treated as background. This technique can create realistic adjacency conditions where two anatomical structures are close together but separated by a thin, low-contrast region. These synthetic boundaries can replicate real-world challenges such as overlapping tissues, indistinct organ interfaces, or pathological invasion of adjacent structures. The erosion module can also support further enhancements, such as selectively applying contrast equalization or shared intensity profiles to the adjacent regions, increasing the difficulty of segmentation. These difficult examples can force the model to learn finer spatial cues, improving its edge localization and object discrimination abilities. As a result, models trained with boundary-aware data can demonstrate improved performance when applied to noisy, ambiguous, or complex real-world medical segmentation tasks.

Various embodiments described herein can be employed to use hardware or software to solve problems that are highly technical in nature (e.g., to facilitate zero-shot domain adaptation of computer vision models for medical image segmentation), that are not abstract and that cannot be performed as a set of mental acts by a human. Further, some of the processes performed can be performed by a specialized computer (e.g., a training server, a cloud-based AI inference engine, or a medical image processing system) for carrying out defined acts related to generating, manipulating, and processing high-resolution synthetic medical images and training machine learning models thereon. For example, such defined acts can include: generating, by a device operatively coupled to a processor, synthetic data that closely approximates a data manifold of medical images; initializing, by the device, a set of encoders with pre-trained weights for a pre-trained model for at least one task; and utilizing, by the device, the generated synthetic data and set of encoders to generate the pre-trained model with pre-trained weights of a pre-trained model for the at least one task.

Such defined acts are not performed manually by humans. Indeed, neither the human mind nor a human with pen and paper can leverage dynamically generated synthetic medical data to simulate texture, contrast, and noise distributions; apply Bézier curve-based object modeling; and update multi-million parameter neural networks using stochastic optimization. Indeed, generating procedural label maps, applying noise functions such as Rician or Perlin noise, and performing on-the-fly model training with high-resolution image inputs are inherently computerized, hardware-and-software-based constructs that simply cannot be meaningfully implemented or trained in any way by the human mind without computers.

Moreover, various embodiments described herein can integrate into a practical application various teachings relating to the automatic adaptation of foundation models to the medical imaging domain. As described above, current model fine-tuning strategies primarily focus on supervised training with real annotated data and do not adequately address the challenge of domain shift, or the scarcity of high-quality medical labels required to train segmentation models from scratch.

Various embodiments described herein can address one or more of these technical problems. In particular, the present inventors recognized that there is a need for a system that enables medical image segmentation models-such as promptable foundation models like SAM—to be effectively trained or adapted for clinical use without requiring any real-world annotated medical datasets.

Furthermore, various embodiments described herein can control real-world tangible devices based on the disclosed teachings. For example, various embodiments described herein can electronically control diagnostic imaging pipelines, medical data visualization systems, and AI-augmented clinical workflow tools. Specifically, various embodiments described herein can output high-quality segmentation maps based on real or synthetic inputs, enabling automated or semi-automated region identification within diagnostic imaging platforms. These processes can improve the speed, reproducibility, and accessibility of medical image interpretation across diverse healthcare environments.

Embodiments described herein can directly control real-world hardware components, such as graphical processing units (GPUs), memory controllers, and imaging modality interface hardware, to automate training loops, image rendering, and ensure real-time inference during deployment. The disclosed system does not merely recite abstract software processes but instead performs concrete computing functions that transform how medical imaging segmentation models are trained, validated, and applied in real clinical settings.

It should be appreciated that the figures and description herein provide non-limiting examples of various embodiments and are not necessarily drawn to scale.

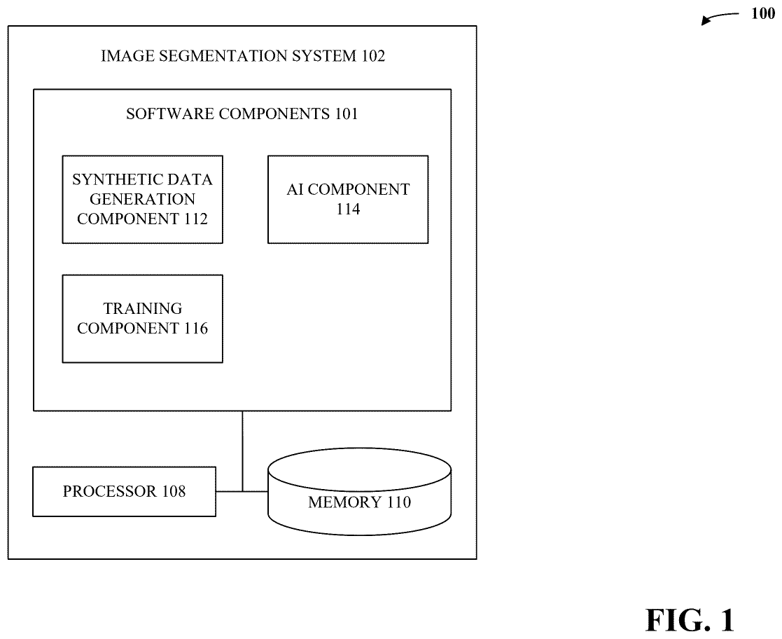

FIG. 1 illustrates a block diagram of an example, non-limiting system 100 that can facilitate automatic product support via generative artificial intelligence and customer interactions. In various embodiments, the image segmentation system 102 can comprise a processor 108 (e.g., computer processing unit, microprocessor) and a non-transitory computer-readable memory 110 that is operably or operatively or communicatively connected or coupled to the processor 108. The non-transitory computer-readable memory 110 can store computer-executable instructions which, upon execution by the processor 108, can cause the processor 108 or other components of the vulnerability management system 102 (e.g., synthetic data generation component 112, artificial intelligence component 114, training component 116) to perform one or more acts. In various embodiments, the non-transitory computer-readable memory 110 can store computer-executable components (e.g., synthetic data generation component 112, artificial intelligence component 114, training component 116), and the processor 108 can execute the computer-executable components.

In various embodiments, the image segmentation system 102 can comprise a synthetic data generation component 112. The synthetic data generation component 112 can generate synthetic data that closely approximates a data manifold of medical images. The synthetic data can emulate structural, statistical, and visual properties representative of real-world medical imaging modalities, including but not limited to computed tomography (CT), magnetic resonance imaging (MRI), ultrasound, and label-free microscopy. The synthetic data generation component 112 can include one or more subcomponents such as a shape-aware module, a boundary-aware module, and a noise and texture library. These subcomponents can be used to construct anatomical structures, simulate spatial adjacency and boundary ambiguity, and apply contrast and noise transformations, resulting in high-variability synthetic images. The component can produce synthetic training images that simulate anatomical variability, modality-specific visual features, and imaging artifacts found in clinical scenarios.

In various embodiments, the synthetic data generation component 112 can generate biologically-inspired synthetic data that reflects anatomical realism, domain-specific structure, and clinically relevant variability. The component 112 can construct foreground and background components using analytical methods that simulate anatomical entities-such as organs, lesions, vessels, or ducts-based on known morphological and radiomic principles. For instance, the generated structures can exhibit realistic shape variation, soft tissue boundary transitions, and modality-specific intensity characteristics. Radiomic features such as shape compactness, texture entropy, edge sharpness, and intensity distribution can be explicitly modeled or controlled during data generation. This biologically-inspired design can ensure that synthetic images are not only visually plausible but also statistically similar to the kinds of image features learned by clinical models trained on real data. Such radiomic-informed generation can improve the generalizability of downstream models trained solely on synthetic inputs.

The shape-aware module of the synthetic data generation component 112 can employ Bézier curve-based object generation to construct diverse anatomical structures. The module can randomly select a number of control points for each Bézier curve to vary the shape complexity. The generated shapes can be blurred using Gaussian filters with randomly selected sigma values to simulate soft tissue boundaries commonly found in medical images. The module can superimpose the generated shapes onto background canvases that are either uniform or include simple geometric phantoms. To simulate contrast differences between anatomical structures and background, the component can adjust intensity values according to a contrast modulation function of the form (1−p)×(m×r), where p is a phantom or background intensity value sampled from a uniform distribution, m is the index of the shape, and r is a randomly sampled variability parameter. This configuration enables the generation of a wide range of anatomical intensities and structural contrasts.

The synthetic data generation component 112 can further include a noise and texture augmentation pipeline configured to apply realistic imaging noise patterns. The component can access a noise library comprising additive Gaussian noise, multiplicative Poisson noise, Rician noise, Perlin noise, and speckle noise. During training, one or more of these noise types can be applied on-the-fly to each generated image to emulate texture and noise conditions encountered in real imaging modalities. Additionally, the boundary-aware module can generate adjacent anatomical structures with thin or ambiguous boundaries. This module can generate multiclass label maps with 10-15 clusters, select a subset of the clusters, and apply erosion operations with randomly selected iteration counts to produce thin boundary regions. The clusters can then be collapsed into binary masks distinguishing foreground from background. Foreground regions can be assigned pixel values from randomized intensity canvases, and background regions can be generated by averaging canvas values and introducing small perturbations.

The synthetic data generation component 112 can be configured to produce synthetic medical images that accurately emulate the visual appearance of real-world scans, including properties such as grayscale distribution, noise level, edge sharpness, and anatomical density. This is achieved by coordinating the operations of the shape aware, contrast and texture, and boundary-aware modules in a pipeline. Each synthetic image can be constructed from scratch by generating synthetic structures, compositing them against a textured or phantom background, and applying contrast modulation and noise patterns in real time. The shape aware synthetic tool component 202 can specifically control the shape complexity of the generated anatomical structures by varying the number of Bézier curve control points. Increasing the number of control points can result in more intricate and irregular shapes, while fewer control points can yield simpler, smoother geometries. This mechanism enables the system to represent both large, well-defined organs (e.g., liver, kidney) and small, irregular structures (e.g., tumors, ducts), giving the trained model exposure to a wide spectrum of segmentation targets. The variability in shape complexity can be essential for training models that generalize across clinical use cases. For example, a model exposed only to regular, easily segmented shapes may fail to correctly identify or delineate pathologies that distort organ boundaries. By generating a mix of simple and complex shapes within the synthetic dataset, the system can simulate a broader distribution of clinical imaging appearances and improve the adaptability of the trained model across patient populations and disease states.

In various embodiments, the image segmentation system 102 can comprise an artificial intelligence component 114.

The artificial intelligence component 114 can use an artificial intelligence model to learn relevant representations of the synthetic data to generate task-specific outputs for an at least one image task. The artificial intelligence model can comprise a general-purpose or pre-trained architecture (e.g., a convolutional neural network, vision transformer, or foundation model) that can be adapted to extract meaningful visual features from biologically-inspired synthetic images. The artificial intelligence component 114 can process the synthetic data to generate latent representations that capture semantic, structural, and radiomic characteristics aligned with the intended task, such as segmentation, classification, or detection. These learned representations can serve as the internal basis for producing task-specific outputs, including but not limited to anatomical masks, region labels, or confidence maps. By leveraging synthetic data tailored to the radiomic properties and spatial configurations of specific imaging tasks, the artificial intelligence model can learn to generalize across diverse imaging modalities without requiring real-world clinical annotations. This configuration can enable the artificial intelligence component 114 to bridge domain-specific priors embedded in synthetic data with the flexible pattern recognition capabilities of a general-purpose model, facilitating downstream performance in medical image analysis.

The artificial intelligence component 114 can utilize a set of encoders. The encoders can be initialized with pre-trained weights for a pre-trained model for at least one task. The set of encoders can be initialized with pre-trained weights for a pre-trained model configured to perform at least one image segmentation task. The encoders can include components from a foundation model architecture, such as the image encoder of the Segment Anything Model (SAM). The pre-trained weights can be derived from training on large-scale natural image datasets comprising diverse objects and segmentation masks. By initializing the encoders with these pre-trained weights, the system can leverage learned representations that generalize well to image features such as edges, textures, and object boundaries, even in domains different from those present in the original training set.

The encoders can be integrated into a segmentation architecture in combination with a decoder and, optionally, a prompt encoder. During training, the system can retain the pre-trained encoder weights in a frozen or semi-frozen state, allowing only the decoder- or selected trainable layers—to be updated based on the synthetic medical training data. This training strategy can preserve the broad representational capacity of the foundation model while adapting downstream segmentation capabilities to medical imaging tasks. The encoder can process synthetic input images at high resolution and generate feature embeddings that are consumed by the decoder to produce segmentation masks aligned with anatomical structures present in the synthetic data.

The set of encoders (e.g., the encoder component) can support on-the-fly training workflows, where synthetic images are dynamically generated and immediately passed through the encoder during each training iteration. This configuration can enable efficient model convergence while avoiding the need to precompute or store large training datasets. The use of pre-trained encoders 114 initialized with general-purpose vision weights can reduce the amount of synthetic data required for effective training and can improve the ability of the resulting model to generalize to real-world medical imaging inputs.

In various embodiments, an artificial intelligence component 114 can use an artificial intelligence model to encode features of biologically-inspired synthetic data for at least one image analysis task. The artificial intelligence component 114 can process synthetic medical images generated by a synthetic data generation pipeline and convert them into high-dimensional feature representations that capture structural, contextual, and radiomic characteristics relevant to the task at hand. The artificial intelligence model can include encoder architectures derived from general-purpose models such as convolutional neural networks, vision transformers, or foundation models trained on large image corpora. By applying the artificial intelligence model to the synthetic data, the component 114 can produce task-relevant embeddings that preserve fine-grained spatial information, texture details, and semantic cues critical for downstream applications such as segmentation, classification, or detection. These encoded features can serve as input to a training pipeline that further refines or adapts the model to perform the target image analysis task using only synthetic training signals, thus avoiding reliance on manually annotated clinical data.

In various embodiments, the image segmentation system 102 can comprise a training component 116. The training component 116 can utilize the synthetic data and the artificial intelligence model to generate a task-specific model for the at least one image analysis task. The training component 116 can receive biologically-inspired synthetic images (e.g., generated to approximate a task-specific data manifold from a radiomic features perspective) and apply them as inputs to the artificial intelligence model to fine-tune or adapt the model toward a targeted clinical outcome. The training component 116 can update selected parameters of the artificial intelligence model (e.g., decoder layers, task-specific heads) based on supervision signals derived from synthetic labels or masks associated with each synthetic image. The training process can employ optimization routines such as stochastic gradient descent or Adam, using loss functions tailored to the intended image analysis task (e.g., Dice loss for segmentation or cross-entropy for classification). By systematically exposing the artificial intelligence model to a high-variability synthetic training set, the training component can enable the emergence of a specialized model that performs reliably in the context of a specific imaging task. This approach can eliminate the need for manually labeled real-world medical data, while promoting generalization to diverse anatomical structures, imaging artifacts, and modality-specific characteristics.

The training component 116 can utilize the synthetic data generated by a synthetic data generation component 112 and the encoded features produced by an artificial intelligence component 114 to generate a task-specific model for at least one image analysis task. The synthetic data from component 112 can include biologically-inspired medical images that reflect task-specific radiomic characteristics, and the encoded features from component 114 can represent abstract representations of those images suitable for downstream learning. The training component 116 can use these inputs as supervision signals to optimize a target model architecture, which can include decoder layers or task-specific heads. In doing so, the training component 116 can facilitate the creation of image analysis models-such as segmentation, classification, or detection models-without reliance on manually labeled real-world data.

The training component 116 can employ a loss function such as Dice loss to measure the similarity between predicted segmentation masks and ground truth synthetic masks. Optimization can be performed using gradient descent-based algorithms, with learning rate schedules such as cosine decay applied over a defined number of training epochs. Each training epoch can consist of a specified number of synthetic image-mask pairs, and the synthetic data can be generated dynamically to ensure high variability and prevent overfitting. The training component 116 can evaluate model performance on a validation subset and select the best-performing model checkpoint based on segmentation accuracy.

The training component 116 can support various training configurations, including freezing the encoder layers initialized with pre-trained weights while training only the decoder, or selectively fine-tuning encoder layers as needed. The component can operate in a low-resource setting with a batch size of one and high-resolution input images, such as 1024×1024 pixels. By leveraging dynamically generated synthetic data and pre-trained encoder representations, the training component 116 can enable efficient domain adaptation of general-purpose foundation models to specialized medical segmentation tasks-without requiring any manually annotated real medical data.

The training component 116 can employ optimization objectives tailored to the image analysis task, such as Dice loss for segmentation or cross-entropy for classification. Optimization can be performed using gradient descent-based methods, including adaptive learning rate schedulers such as cosine decay. Each training epoch can include either dynamically generated synthetic image-mask pairs from component 112 or pre-generated and stored examples. This dual capability allows the training component 116 to support both on-the-fly augmentation and reproducible, benchmarkable datasets. Model performance can be validated on held-out synthetic samples, and a best-performing checkpoint can be selected using criteria such as intersection-over-union or F1 score.

The training component 116 can further support modular training workflows. For instance, the artificial intelligence component 114 can be held fixed while the task-specific decoder is trained, or both components can be fine-tuned jointly. The system can support training under resource-constrained settings by allowing small batch sizes and high-resolution inputs. By combining biologically-informed synthetic data from component 112 with encoded representations from component 114, the training component 116 can generate robust, task-specific models that generalize well across anatomical variation, imaging modality, and clinical context.

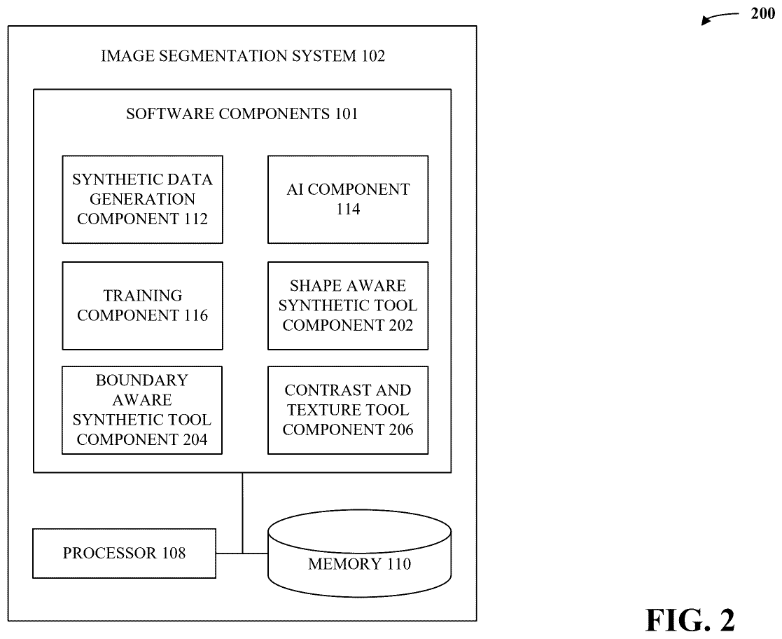

Next, FIG. 2 illustrates a block diagram of an example, non-limiting system 200 that facilitates automatic product support via generative artificial intelligence and customer interactions. As shown, the system 200 can, in some cases, comprise the same components as the system 100, and can further comprise a shape aware synthetic tool component 202, a boundary aware synthetic tool component 204, and a contrast and texture tool component 206.

The shape aware synthetic tool component 202 can employ Bézier curve-based object generation to construct a diverse set of synthetic anatomical structures used to train the artificial intelligence model within component 114. Bézier curves can provide a mathematically defined way to generate smooth and continuous contours based on control points. The component 202 can randomly vary the number and placement of Bézier control points to produce shapes of differing geometric complexity. These shapes can simulate anatomical features such as organs or lesions, and can be embedded into foreground masks overlaid on uniform or phantom-rich background canvases. To further increase anatomical variability, the component can support geometric transformations such as rotation, scaling, and translation. In some configurations, shape templates based on real anatomical statistics can seed the generation process, enabling population-level variation due to age, pathology, or biological diversity. The resulting masks can be stored as binary or multiclass label maps and used downstream for generating synthetic image-mask pairs.

The boundary aware synthetic tool component 204 can simulate anatomical structures that are closely packed and share ambiguous or thin boundaries. These boundary conditions frequently occur in clinical imaging contexts, such as between organs in abdominal CT or soft tissue structures in MRI. Component 204 can construct multiclass label maps with 10-15 clusters, then randomly select a subset and apply erosion operations to define narrow boundary zones. The number of erosion iterations and kernel size can be randomly sampled to ensure variable boundary thicknesses. Following erosion, the clusters can be merged into a foreground-background binary mask or retained as multiclass annotations. This tool can simulate realistic structural adjacency, including overlap, partial occlusion, or shared intensity features, thereby helping the artificial intelligence model learn fine-grained boundary delineation. The boundary-aware tool 204 can challenge the model with difficult edge cases, improving its ability to generalize to ambiguous or low-contrast real-world data.

Component 204 can also include an erosion module specifically configured to generate morphological variations in boundary separation. This module can use random sampling to determine erosion parameters per cluster, providing fine-grained control over how boundary thicknesses are distributed in the training data. After the erosion process, adjacent clusters can be preserved as foreground, while the space between them becomes background, allowing for realistic but difficult-to-segment transitions. Additional processing can include adjusting intensities or applying shared texture gradients across adjacent structures to further increase segmentation complexity. These synthetic conditions can help train task-specific models that are more resilient to imaging artifacts, anatomical overlap, and modality-specific ambiguities.

The contrast and texture tool component 206 can enhance synthetic image realism by simulating the grayscale intensity dynamics, blurriness, and noise characteristics common to medical imaging modalities. It can apply multiple image augmentation operations such as Gaussian noise, Gaussian blurring, and phantom intensity overlays to replicate modality-specific acquisition artifacts. Gaussian noise can be added with varying standard deviation levels to emulate low-signal environments, sensor variability, or electronic noise in imaging systems such as ultrasound or low-dose CT. Gaussian blurring can be used to degrade the sharpness of anatomical edges, simulating phenomena such as motion blur, low spatial resolution, or partial volume effects. The tool can vary the blurring kernel size and sigma values to apply different levels of edge softness to each image, reinforcing the model's ability to localize structures under uncertain conditions. In some instances, the background canvas can be overlaid with circular phantom shapes or smooth gradients to represent the homogeneous appearance of soft tissue regions commonly found in CT and MR scans. These phantoms can serve as background anchors that enhance the perceived realism of spatial context within the image. The component can also apply controlled intensity modulation across anatomical structures using a formula such as (1−p)×(m×r), where p is a phantom or background intensity value sampled from a uniform distribution, m represents the shape index, and r is a random number from the interval [−0.2, 0.2]. This modulation introduces contrast differences across shapes and between foreground and background, replicating the subtle grayscale variations characteristic of many medical modalities. By dynamically varying these parameters, the contrast and texture tool component 206 can produce a synthetic dataset with high visual diversity and realistic appearance, enabling robust model training without relying on real-world annotated medical data.

The contrast and texture tool component 206 can include a noise library configured to introduce multiple types of noise to the synthetic training images. This noise library can be used to simulate real-world acquisition noise present in different imaging modalities. The available noise types can include Poisson noise, which models photon shot noise in low-light conditions; Rician noise, which arises in magnitude-reconstructed MR images; speckle noise, typical in ultrasound imaging; and Perlin noise, which can add organic-looking texture variations. These noise types can be applied alone or in combination to foreground, background, or the entire image canvas. During training, the system can randomly select one or more noise models and apply them with varying strength and spatial distribution. This randomization can occur on a per-image basis or at the patch level, ensuring that the synthetic data distribution remains broad and non-redundant. The ability to inject multiple types of noise across spatial and temporal dimensions can help the model become robust to noise-induced segmentation artifacts and generalize well to real-world imaging conditions. The noise library can be tightly integrated with other synthetic data generation modules to ensure that noise is applied after contrast modulation and structural rendering, thereby preserving the semantic integrity of the synthetic annotations. The component can also be configured to maintain consistent noise levels across multiple training epochs or introduce gradual noise escalation strategies, which can support curriculum-based learning workflows for more stable model convergence.

In some implementations, the contrast and texture tool component 206 can further include generative artificial intelligence methods, such as diffusion models, variational autoencoders, or GAN-based techniques, to synthesize or modify texture regions or contrast zones within synthetic images. These generative methods can learn latent distributions of medical image patches and apply probabilistic sampling to generate fine-grained texture variations that mimic modality-specific noise and background characteristics. For instance, a generative AI subcomponent can generate background textures that replicate tissue homogeneity or create localized intensity patterns that simulate pathology-like features. These generative enhancements can operate alongside traditional augmentation pipelines to increase realism, texture diversity, and radiomic fidelity within the synthetic dataset.

Additionally, in various embodiments, the synthetic data generation component 112 can include a contrast modulation engine that assigns intensity values to foreground and background regions of each label map using randomized functions. For example, each anatomical region can be assigned an intensity canvas sampled from a normal or uniform distribution, while background regions can be calculated as smoothed or averaged variants of those intensities with small perturbations. These randomization strategies can emulate real-world grayscale variability across modalities such as MRI, CT, and ultrasound, where tissue classes may overlap in intensity space. By modulating pixel intensities independently for each structure, the system 102 can create challenging segmentation scenarios and increase the model's robustness to low-contrast interfaces.

The artificial intelligence component 114 can include a general-purpose artificial intelligence model configured to encode structural and radiomic features from the synthetic data generated by component 112. The encoded features can preserve anatomical context, shape properties, edge gradients, and pixel-level texture, enabling downstream components to process and learn from semantically meaningful patterns. The artificial intelligence model within component 114 can be implemented using deep learning-based encoders such as convolutional backbones, transformer-based vision encoders, or hybrid architectures, and can output embeddings suitable for segmentation, classification, or multimodal fusion. These encoded outputs can be passed directly to the training component 116, which can use them to produce a task-specific model fine-tuned for one or more clinical tasks.

In some implementations, the artificial intelligence model can receive structured input prompts that include task-specific guidance, such as class labels, bounding boxes, or regions of interest. These prompts can be combined with the synthetic image as part of a multi-channel input tensor or provided as tokenized input depending on the model architecture (e.g., transformer-based systems). The model can thereby learn context-aware representations tailored to clinical objectives like tumor detection, organ segmentation, or region-level classification.

In various embodiments, the system can incorporate evaluation metrics during or after training to assess model performance on synthetic or real-world validation datasets. For segmentation tasks, metrics such as Dice similarity coefficient (DSC), Intersection over Union (IoU), precision, recall, and Hausdorff distance can be used to measure boundary accuracy, region overlap, and structural correctness. For classification tasks, metrics such as area under the receiver operating characteristic curve (AUC-ROC), F1 score, and accuracy can be applied. These evaluations can inform early stopping criteria and hyperparameter optimization within the training component.

In various aspects, the training component 116 can implement encoder-decoder architectures, where the encoder is derived from a general-purpose artificial intelligence model and the decoder is optimized using synthetic supervision. The decoder can include convolutional blocks, transformer layers, or multi-scale fusion mechanisms designed to interpret high-dimensional embeddings and produce dense predictions. Training can proceed using mini-batch gradient descent, AdamW optimization, and cosine annealing of the learning rate. The model can be trained from scratch or fine-tuned, depending on the initialization and complexity of the downstream task.

In some embodiments, the synthetic dataset can include image-label pairs with varying anatomical shapes, boundary configurations, intensity distributions, and imaging noise profiles. Class balancing strategies can be employed during generation to ensure representation of rare but clinically significant structures. Sampling techniques such as rejection sampling, weighted distributions, or curriculum learning schedules can be applied to prioritize harder samples at later training stages, improving generalization to real-world tasks.

In addition to shape and noise modeling, the system can simulate radiomic feature distributions found in clinical datasets, such as texture entropy, contrast energy, and gray-level co-occurrence statistics. These features can be controlled via parameterized texture maps, intensity functions, and boundary gradient distributions. The synthetic data generation component 112 can be configured to match the statistical properties of real patient cohorts based on curated reference distributions, thereby aligning the synthetic training domain with target clinical applications.

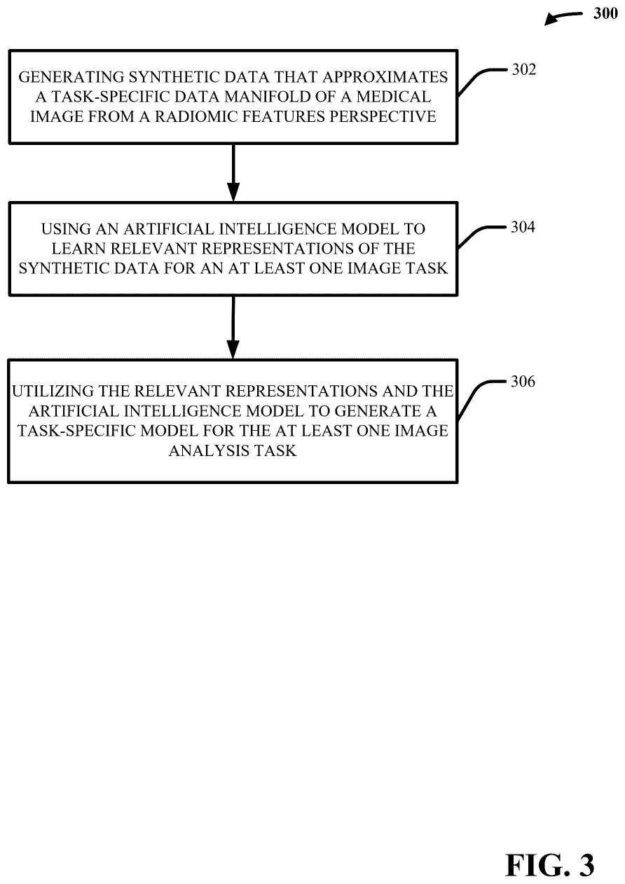

Next, FIG. 3 illustrates a flow diagram of an example, non-limiting computer-implemented method 300 that can facilitate automatic product support via generative artificial intelligence and customer interaction in accordance with one or more embodiments described herein.

In various embodiments, act 302 of FIG. 3 can include generating, by a system (e.g., via synthetic data generation component 112) operatively coupled to a processor (e.g., processor 108), biologically-inspired synthetic data that approximates a task-specific data manifold of a medical image from a radiomic features perspective. This synthetic data can be procedurally generated to emulate structural, statistical, and contextual characteristics observed in medical images across modalities such as CT, MRI, and ultrasound. The data generation process can incorporate shape, contrast, boundary, and texture variations based on domain-specific radiomic properties, including intensity histograms, edge sharpness, and local texture entropy. Anatomical structures can be generated using Bézier curve-based geometry, and combined with intensity modulation functions that simulate realistic grayscale distributions and tissue contrast. Noise models such as Poisson, Rician, Perlin, and speckle noise can be added to further enhance realism. The system can support both on-the-fly generation during training and offline dataset generation, including saving each synthetic image along with metadata describing all parameters used in its creation. This dual-mode functionality allows the method to support reproducibility, benchmarking, and scalable training without reliance on annotated real-world medical data. In various embodiments, the synthetic data can be biologically-inspired synthetic data that closely approximates a data manifold of a specific purpose/task pertaining to medical images from radiomic features perspective. This can be achieved by analytically compositing the image's foreground and background components in accordance with task-specific requirements, ensuring that both structural and contextual features relevant to the intended application are realistically modeled.

In various embodiments, act 304 of FIG. 3 can include using an artificial intelligence model (e.g., via artificial intelligence component 114) to learn relevant representations of the synthetic data to generate task-specific outputs for at least one image task. The artificial intelligence model can include a general-purpose encoder architecture, such as a convolutional neural network or vision transformer, optionally pre-trained on a large-scale corpus of natural or medical images. When applied to biologically-inspired synthetic data, the model can learn internal representations that capture radiomic attributes such as morphological variation, boundary ambiguity, and intensity heterogeneity. These representations can encode semantic and spatial information necessary for solving downstream image tasks, including segmentation, classification, detection, or registration. In some implementations, the artificial intelligence model can be partially or fully fine-tuned on synthetic supervision signals, enabling the system to generate accurate task-specific outputs without reliance on real-world annotations. The model's ability to extract and refine these representations from synthetic inputs can support transferability to real clinical imaging data, facilitating zero-shot or few-shot performance in practical applications.

In various embodiments, the artificial intelligence model can process the synthetic data to generate task-specific outputs in the form of intermediate feature representations that support downstream training workflows. These outputs can comprise latent embeddings or structured vectors that encode spatial, radiomic, and semantic characteristics of the synthetic data, including intensity gradients, shape contours, edge sharpness, and inter-structure relationships. By transforming raw synthetic images into these learned feature spaces, the artificial intelligence model can facilitate the extraction of abstract representations tailored to one or more image analysis tasks, such as segmentation, classification, or detection. The model can include general-purpose encoders (e.g., convolutional neural networks or vision transformers) optionally initialized with pre-trained weights from large image corpora. These encoders can be fixed during task-specific training or fine-tuned alongside decoder layers or task heads. The resulting task-specific outputs can enable construction of robust, targeted models capable of adapting general-purpose representations to the particular requirements of a given medical imaging task.

In various embodiments, act 306 of FIG. 3 can include utilizing, by a training component (e.g., training component 116), the synthetic data and the artificial intelligence model to generate a task-specific model for at least one image analysis task. The training component can operate by applying the artificial intelligence model to the synthetic data to extract meaningful representations, and then refining or adapting the model's parameters to specialize it for a designated image task such as segmentation, classification, or detection. This training pipeline can incorporate a decoder or task-specific head that processes intermediate representations learned by the artificial intelligence model and produces task-aligned outputs. Supervision can be provided through synthetic labels generated alongside the data, with optimization guided by loss functions such as Dice loss or cross-entropy. Training can leverage either dynamically generated synthetic samples, precomputed datasets, or a hybrid of both. Optimization algorithms such as stochastic gradient descent and learning rate schedules like cosine decay can be employed to efficiently converge on performant task-specific parameters. By training directly on biologically-inspired synthetic data using a general-purpose model architecture, the system can produce task-specialized models that generalize well to real-world clinical imaging tasks without relying on manually labeled patient data.

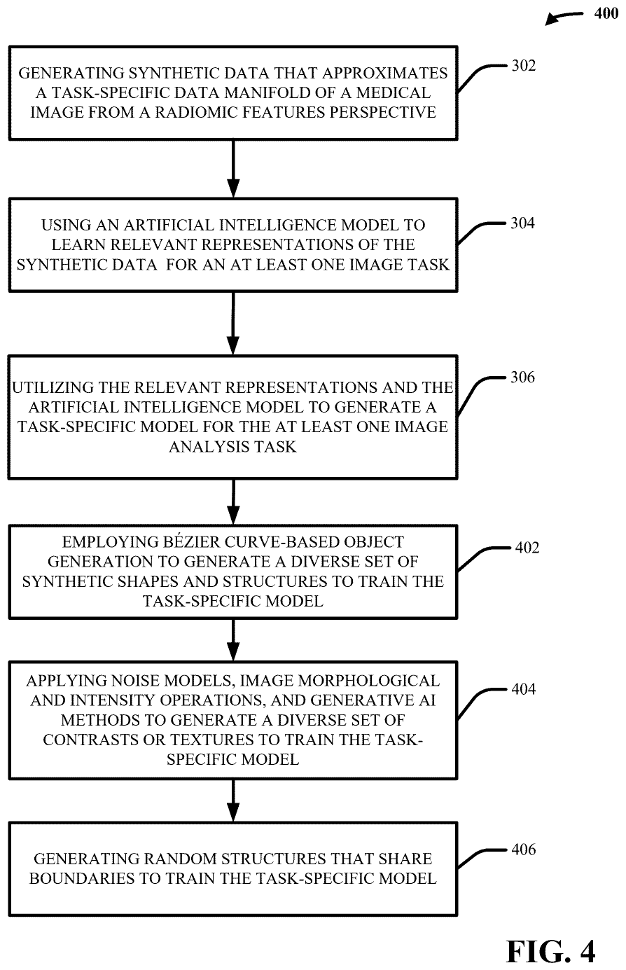

Next, FIG. 4 illustrates a flow diagram of an example, non-limiting computer-implemented method 400 that can facilitate automatic product support via generative artificial intelligence and customer interaction in accordance with one or more embodiments described herein. Repeated descriptions of like elements have been omitted for brevity. As shown, method 400 includes acts 302, 304, and 306, which have been previously described with respect to FIG. 3. Following those acts, method 400 includes additional operations related to generating and diversifying the synthetic training data using specific modules within the synthetic data generation component.

In various embodiments, act 402 can include employing Bézier curve-based object generation to construct a diverse set of synthetic anatomical structures. A shape aware synthetic tool component can execute this operation on a computing device operatively coupled to a processor. Bézier curves can be defined by a variable number of control points, and the system can sample both the quantity and placement of control points to produce objects of differing geometric complexity and curvature. These shapes can simulate both regular and irregular anatomical forms, including organs, lesions, and tissues. The generated structures can be rendered as foreground regions overlaid on uniform, textured, or phantom-rich background canvases. Post-processing operations such as rotation, scaling, translation, and intensity modulation can further enhance anatomical variability. In some configurations, the generation process can be guided by statistical shape templates derived from real medical datasets, allowing the synthetic structures to capture population-level morphological patterns. The resulting masks can be stored and paired with synthetic images during model training.

In various embodiments, act 404 can include applying image augmentation operations to generate a diverse range of contrast and texture characteristics representative of real-world medical modalities. This operation can be performed by a contrast and texture tool component configured to simulate intensity dynamics, noise properties, and structural detail common to medical imaging environments. The system can apply Gaussian blurring, phantom overlays, and intensity modulation across anatomical regions to simulate soft tissue indistinctness, motion artifacts, or partial volume effects. Noise models such as Poisson noise, Rician noise, speckle noise, and Perlin noise can be introduced to different parts of the image with random magnitude and spatial configuration. The tool can also modulate the contrast between foreground and background regions using randomized intensity functions, such as a formula of the form (1−p)×(m×r), where p is a sampled background intensity value, m is an object index, and r is a variability term. In some cases, the system can incorporate generative AI techniques to simulate high-level image textures or modality-specific patterns. These contrast and texture variations can be introduced dynamically during training or embedded in pre-generated synthetic datasets, enriching the diversity and realism of the synthetic image distribution.

In various embodiments, act 406 can include generating anatomical structures that share thin or ambiguous boundaries to simulate adjacency scenarios encountered in clinical imaging. A boundary-aware synthetic tool component can be used to execute this operation. The system can begin by generating multiclass label maps containing clusters that represent anatomical regions. A subset of these clusters can be selected, and morphological erosion operations can be applied with randomly determined iteration counts to reduce their extent and form narrow separation zones. After erosion, the remaining clusters can be retained as foreground and the intervening space treated as background, creating binary or multiclass masks that simulate close-contact anatomical relationships. These masks can reflect real-world challenges such as overlapping organs, low-contrast edges, or tumor invasion into neighboring tissues. To further increase difficulty, the system can apply shared intensity profiles or gradient textures across adjacent structures, making segmentation boundaries harder to distinguish. These conditions can promote finer spatial discrimination during training and improve the model's ability to generalize to complex or ambiguous real-world imaging scenarios.

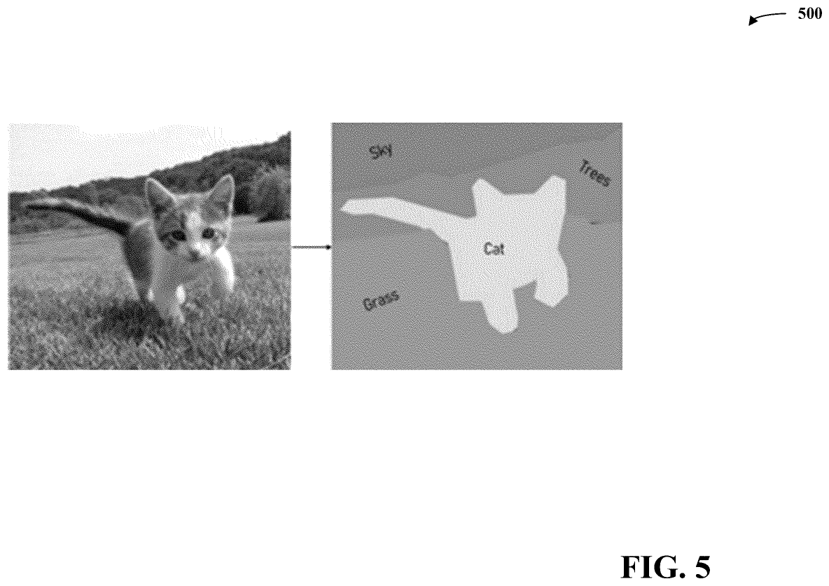

Next, FIG. 5 illustrates image segmentation 500 in accordance with one or more embodiments described herein. The figure shows an example input image (left) and a corresponding segmented output (right). In this example, the input image is a color photograph of a cat walking on a grassy field with a treeline and blue sky in the background. The image includes multiple distinct regions, such as the cat in the foreground, the grass underfoot, the sky above, and trees lining the horizon. The segmented output represents a pixel-wise classification of the input image, in which each pixel has been assigned a class label corresponding to a specific semantic region. As shown, the cat is isolated as a single labeled region (“Cat”) in yellow, with the surrounding areas labeled as “Grass” (green), “Sky” (blue), and “Trees” (purple). The segmentation mask is overlaid with distinct colors to visually differentiate each class, and the labels identify the type of object or background region each segment corresponds to. This example demonstrates the ability of a segmentation model-such as one trained using the synthetic data generation pipeline described in prior embodiments—to accurately identify and delineate semantically meaningful regions within an input image. While FIG. 5 depicts a natural image for illustrative clarity, similar segmentation can be applied to medical images, where the identified regions may correspond to organs, tissues, lesions, or other anatomical structures. The segmentation mask can be generated based on encoder-decoder architectures using positive prompts, as discussed in connection with previous figures, and can be used for automated analysis, diagnostic support, or interactive editing in downstream applications.

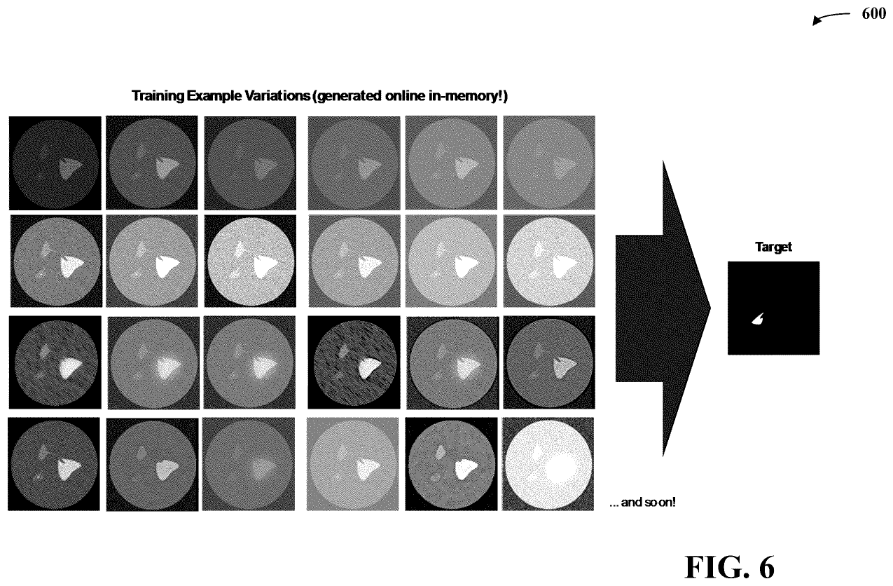

Next, FIG. 6 illustrates example variations of training images with respect to a target in accordance with one or more embodiments described herein. The figure demonstrates how a synthetic data generation pipeline can produce numerous randomized instances of a base training example, each paired with a consistent ground truth segmentation mask. This capability enables dynamic and memory-efficient training by generating new samples on-the-fly rather than relying on static, precomputed datasets. On the left side of FIG. 6, multiple variations of a synthetic training image are shown. These variations include differences in noise, contrast, blur, texture, and intensity modulation. Each image contains a similar underlying anatomical structure but appears visually distinct due to randomized augmentation parameters applied during generation. Some examples show heavy Gaussian noise, others exhibit different levels of background intensity, sharpness, or synthetic phantom artifacts. Despite these differences, all variations maintain the same general spatial layout and segmentation target. To the right of the image grid, a bold arrow points to a binary segmentation mask labeled “Target.” This mask remains consistent across all image variants, indicating the segmentation objective for training. Each generated image can be used as an input while the target mask serves as the supervision label during model optimization. Together, these examples highlight the pipeline's ability to simulate a wide range of clinically plausible input conditions while maintaining a stable and well-defined learning objective. This synthetic variation promotes generalization and robustness in trained segmentation models without requiring any real-world annotated medical data.

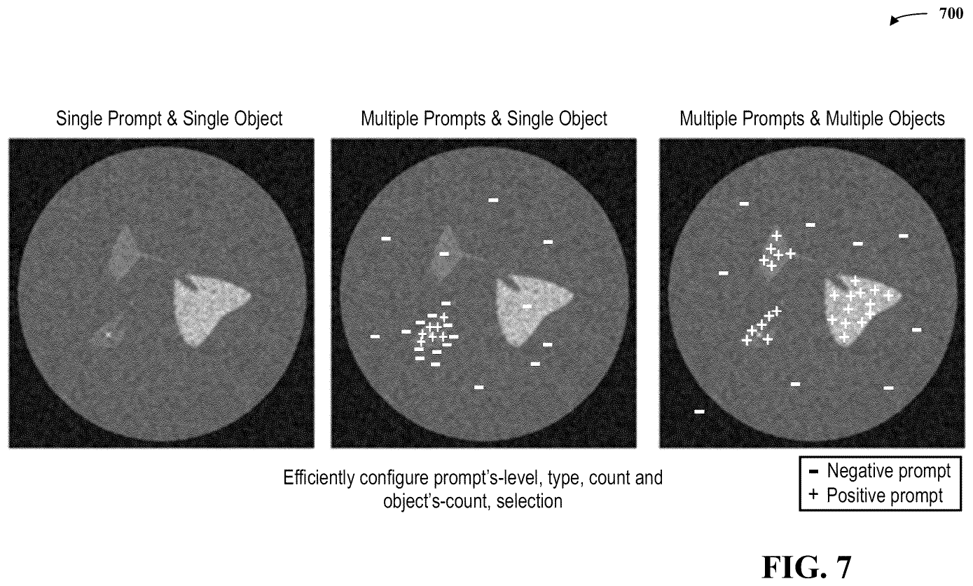

Next, FIG. 7 illustrates a single prompt & single object; multiple prompts & single object; and multiple prompts & multiple objects in accordance with one or more embodiments described herein. The figure showcases three representative input configurations that reflect different prompting strategies used in training or evaluating a promptable segmentation model. These strategies vary in terms of the number of positive and negative prompts provided to the model and the number of target objects to be segmented within a synthetic medical image. In the leftmost panel, labeled “Single Prompt & Single Object,” a synthetic image contains one anatomical structure with a single positive prompt, marked by a cross, placed near the centroid of the object. This setup simulates a simple interactive segmentation scenario where the model is expected to segment a region of interest using minimal user input. In the center panel, labeled “Multiple Prompts & Single Object,” the same structure is associated with several crosses (positive prompts) distributed across the object and dashes (negative prompts) placed outside the object. This configuration introduces additional information to help the model refine its segmentation, particularly when the object is large, irregularly shaped, or surrounded by distractors. In the rightmost panel, labeled “Multiple Prompts & Multiple Objects,” multiple anatomical structures are present within the image. Each structure is associated with its own set of positive prompts, while negative prompts remain distributed outside the relevant regions. This more complex prompting strategy allows the system to segment multiple target regions simultaneously and trains the model to associate prompt-object correspondence accurately. Together, these configurations demonstrate the flexibility of the synthetic data pipeline to generate training samples with varied prompt counts, spatial placements, and segmentation complexity. This variability helps the model generalize across different user interaction styles and segmentation tasks, supporting real-world applicability in interactive medical imaging workflows.

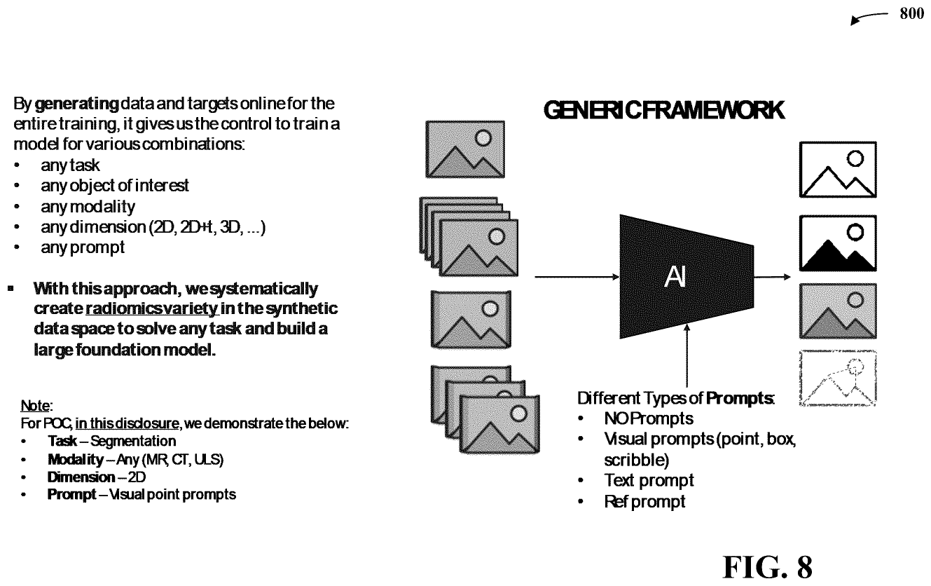

Next, FIG. 8 illustrates a framework for various embodiments in accordance with one or more embodiments described herein. FIG. 8 provides a conceptual overview of how synthetic data can be dynamically generated and utilized in a generalized training pipeline to support model development across a wide range of tasks, modalities, and prompt configurations. This framework is designed to provide high flexibility and control, enabling the training of adaptable, robust foundation models. On the left side of the figure, multiple stacks of images represent synthetic training data generated on-the-fly. These images can be created in real time during training using a synthetic data generation pipeline, eliminating the need for large precomputed datasets. Each image can be rendered with random variations in anatomy, modality simulation, noise, contrast, and prompt configuration. This allows the training system to span a broad range of possible data conditions. As indicated in the accompanying text, this framework supports any task (e.g., segmentation, classification), any object of interest, any modality (e.g., MRI, CT, ultrasound), any dimension (e.g., 2D, 3D, time series), and any type of prompt. The central component labeled “AI” represents a learning model, such as a segmentation model, that receives synthetic training images and associated prompts as input. These prompts can include visual cues (e.g., points, boxes, scribbles), textual descriptions, or reference examples. The prompt variety enables interactive training and supports the development of models that generalize across input styles. Output examples shown on the right side of the figure include different types of segmentation masks and contour overlays, reflecting the model's predictions under various training configurations. The framework emphasizes that by generating data and targets online during the full training lifecycle, developers retain control over how the model is exposed to variability. This includes controlling the prompt type, object distribution, image complexity, and augmentation strategy. As noted, this disclosure demonstrates the approach for the segmentation task using 2D images and visual point prompts across any imaging modality. The framework supports the creation of radiomics diversity in synthetic data, enabling scalable and general-purpose foundation model training.

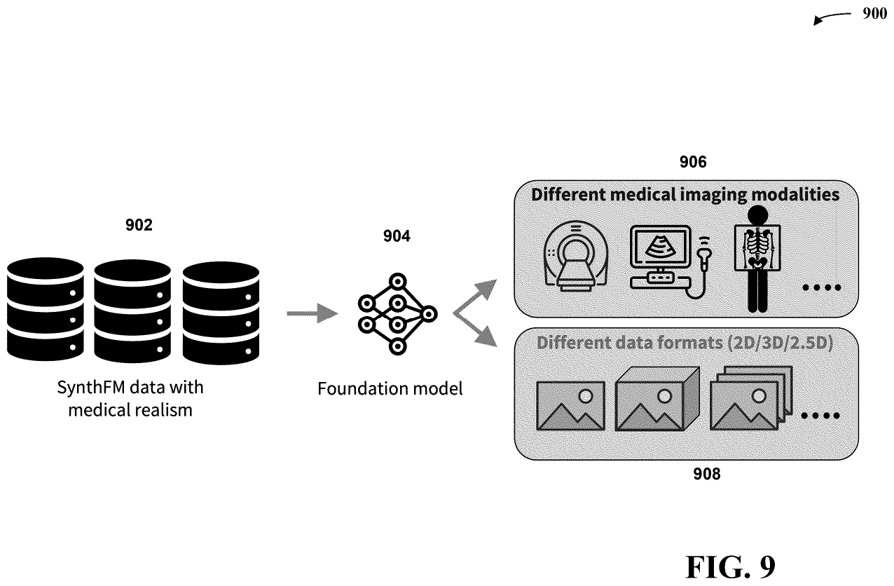

Next, FIG. 9 illustrates a system-level overview of how synthetic data with medical realism can be used to train a foundation model that generalizes across a range of clinical imaging contexts. On the left side of the figure, a set of storage icons 902 labeled “SynthFM data with medical realism” represents a collection of synthetic training datasets generated using anatomically and visually accurate modeling tools. These datasets simulate the properties of real-world medical images, including shape variation, boundary ambiguity, and modality-specific contrast and noise. The synthetic datasets feed into a central neural network 904 labeled “Foundation model.” This model can be pre-trained using the synthetic data, allowing it to learn generalizable visual representations suitable for segmentation, classification, and other image analysis tasks. The bidirectional arrows between the foundation model 904 and the two right-hand components indicate interoperability and adaptability. The top right portion of the figure includes “Different medical imaging modalities” 906, depicting CT, ultrasound, and X-ray imaging devices, among others. This illustrates that the foundation model, once trained, can be adapted to handle various imaging modalities encountered in clinical settings. The bottom right “Different data formats (2D/3D/2.5D)” 908 includes flat images, volumetric stacks, and intermediate data formats. This portion highlights the model's flexibility in handling data of varying dimensional complexity. Together, the components of FIG. 9 convey how synthetic data can be strategically used to create a modality- and format-agnostic foundation model for medical image analysis.

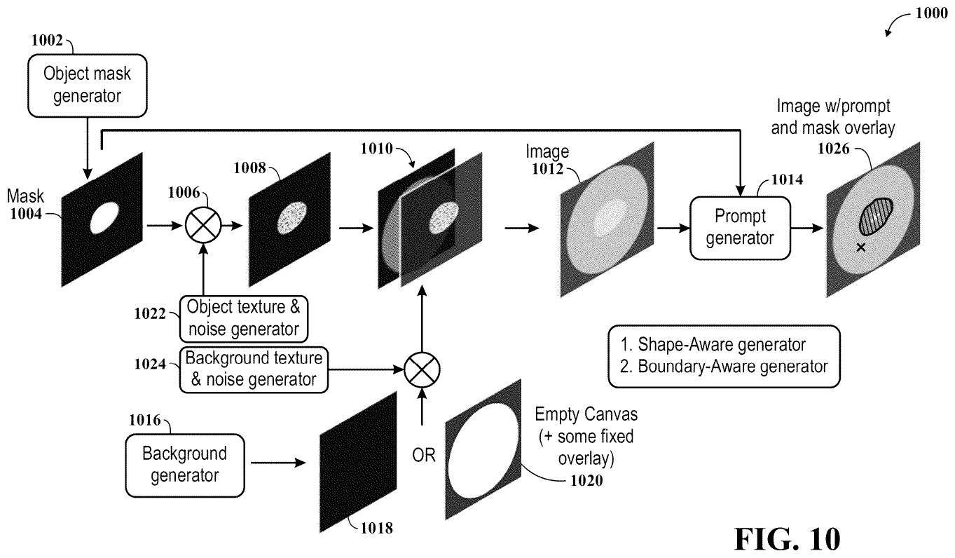

Next, FIG. 10 illustrates a detailed block diagram 1000 of a synthetic image generation and annotation pipeline in accordance with one or more embodiments described herein. On the left side, an object mask generator 1002 can produce a segmentation mask 1004 that defines the spatial extent and shape of a foreground structure. This mask can serve as a binary template to control subsequent image generation operations. The segmentation mask 1004 can be passed to an object texture and noise generator 1022, which can assign intensity values and stochastic noise patterns representative of real-world organ textures. The output from this generator can be blended with the binary mask using a compositing operation 1006 to produce a textured foreground object 1008. In parallel, a background generator 1016 can produce a spatially consistent background image 1018, or alternatively, an empty canvas with a fixed overlay 1020. This background can be enhanced by a background texture and noise generator 1024, which can simulate modality-specific image artifacts, including Gaussian noise, Rician noise, or speckle. The textured foreground 1008 and background 1018/1020 can be composited using a second blending operation to yield a synthetic medical image 1010. This full image 1012 can include realistic anatomical foregrounds embedded within clinically plausible backgrounds. A prompt generator 1014 can then create one or more prompts-such as visual points, bounding boxes, or region cues—that can be applied to the image for training purposes. The final output 1026 can include a fully composed synthetic image with both a visible mask overlay and prompts, supporting multi-modal supervision signals for training. Additionally, as shown in the bottom right, a shape-aware or boundary-aware generator (1, 2) can be used as the initial mask generator 1002 to yield structural or adjacency complexity in the generated object mask 1004.

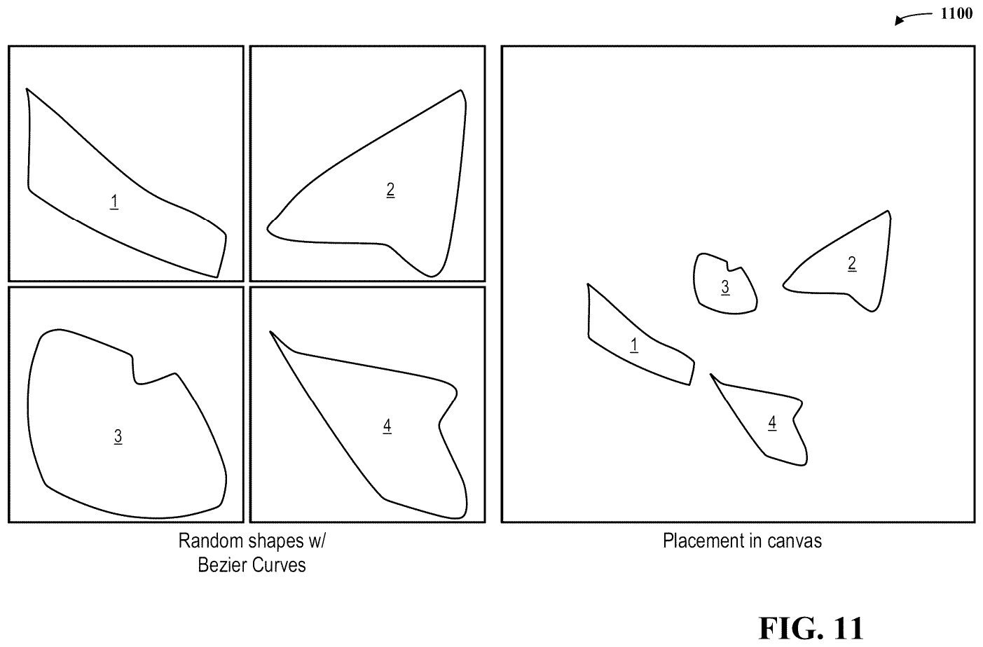

Next, FIG. 11 illustrates an example synthetic shape generation process that can employ Bézier curves to produce anatomically inspired geometries and spatial configurations, in accordance with one or more embodiments described herein. On the left side of the figure, four example shapes are shown, each generated using randomized Bézier curve configurations. These shapes can vary in complexity, curvature, and orientation, reflecting a wide distribution of plausible anatomical structures. A Bézier-based generation process can be used to ensure non-linearity and structural diversity, which can improve model generalization to atypical or irregular shapes encountered in clinical scenarios. On the right side of the figure, the randomly generated shapes can be placed within a canvas to form a composite label map. This placement process can ensure that shapes are distributed without overlap, or can be configured to enforce adjacency and boundary interaction, depending on training objectives. Placement can incorporate spatial constraints, such as minimum distance between objects, boundary contact rules, or clustering of shapes into anatomical-like groupings. The figure demonstrates how random Bézier-generated shapes can be systematically positioned to produce multi-instance segmentation targets that simulate the complexity of real-world medical scenes.

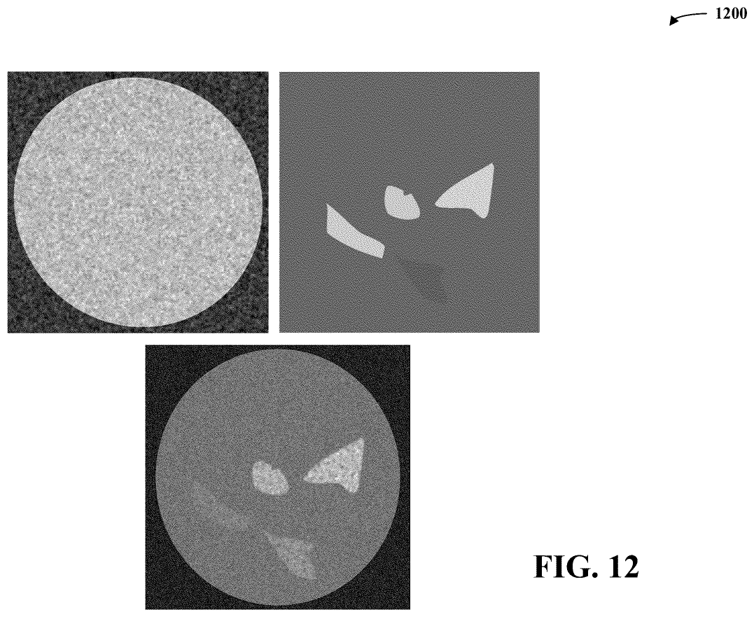

Next, FIG. 12 illustrates a synthetic image generation workflow that can combine background textures with label map overlays to produce realistic training samples in accordance with one or more embodiments described herein. In the top left portion of the figure, a grayscale background image can be observed, generated using texture and noise modeling techniques to emulate radiological scan properties such as speckle, Gaussian, or Perlin noise. This synthetic background can serve as a foundation for composing medically realistic training images. The top right portion of the figure displays a label map with multiple non-overlapping shapes generated using Bézier curves and positioned within a synthetic canvas. These shapes can represent synthetic anatomical structures, each labeled distinctly to facilitate supervised training for segmentation tasks. The label map can include color-coded classes, although in deployment the color coding can be converted to discrete class indices. The bottom portion of the figure illustrates the final composite image created by combining the synthetic background with the shape label map. The merged result exhibits varying contrast and texture within each object and the surrounding background, thereby mimicking the visual properties of real-world medical scans. This approach can be used to generate large quantities of diverse, annotated training data that closely approximate real clinical image distributions without requiring manual labeling.

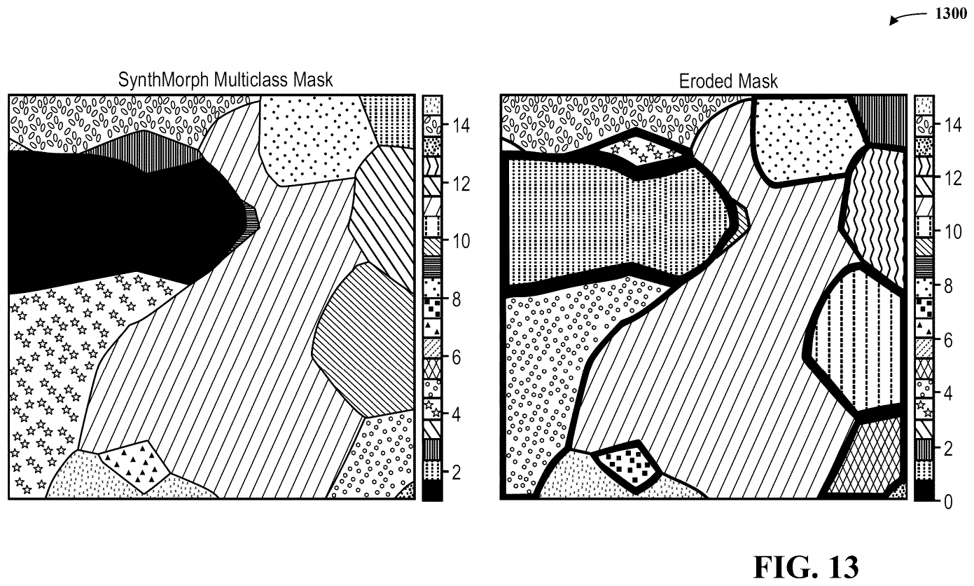

Next, FIG. 13 illustrates the effect of a boundary-aware shape generation module on a multiclass segmentation mask in accordance with one or more embodiments described herein. The left side of the figure shows a SynthMorph multiclass mask that can be generated using synthetic label creation techniques. In this visualization, each distinct region corresponds to a unique anatomical class, and the shared boundaries between adjacent structures can be observed as direct transitions without explicit separation. This form of labeling can approximate tissue adjacency in medical imagery, but it can lack explicit inter-class boundary definition. The right side of the figure presents a corresponding eroded mask, which can be generated by applying a boundary-aware synthetic tool. This tool can introduce boundary separation by performing controlled erosion operations on a subset of adjacent structures. As a result, narrow boundary gaps can be introduced between adjacent class regions, providing more realistic class separation and mimicking the appearance of physiological boundaries in clinical scans. This erosion-based augmentation can improve model training by forcing the model to resolve boundaries with greater precision. Such boundary-aware preprocessing can be particularly advantageous in segmentation tasks where adjacent tissues may have similar visual textures or low-contrast edges. The shape-aware module, in conjunction with this erosion-based transformation, can support more anatomically faithful synthetic segmentation data generation.

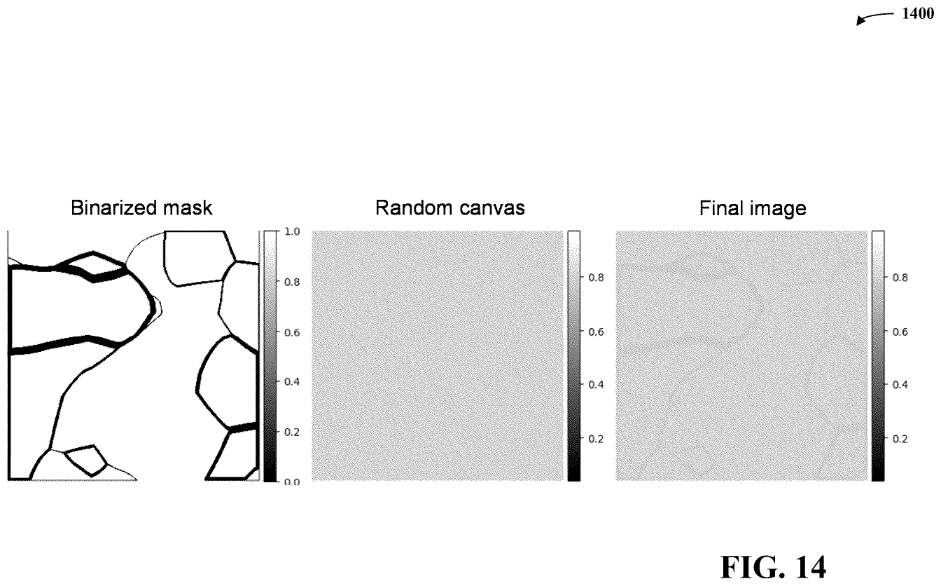

Next, FIG. 14 illustrates an example workflow for generating synthetic medical image textures using a boundary-aware binarized mask and random background canvas in accordance with one or more embodiments described herein. On the left side of the figure, a binarized mask labeled “Binarized mask” can be generated from a multiclass label map. This mask can highlight inter-class boundaries by isolating the pixels that fall on or near the borders between adjacent regions. The thickened boundaries in the mask can represent separation zones that introduce realistic anatomical discontinuities into the synthetic image generation process. In the middle of the figure, a grayscale panel labeled “Random canvas” can serve as a base texture layer. This canvas can be constructed using noise distributions (e.g., Gaussian, Perlin) or real image-derived statistics to simulate a range of clinical background intensities and textures. The random canvas can represent an unstructured, modality-agnostic image domain onto which synthetic anatomical structures can be overlaid. On the right side of the figure, a resulting image labeled “Final image” can be produced by applying the binarized boundary mask to the random canvas. This composite image can contain both background texture and enhanced visual separation at object borders. The resulting visual artifact introduces boundary-aware realism while maintaining stochastic variability across the field of view. This pipeline can support the training of segmentation or detection models that require robust generalization to subtle contrast differences and ambiguous inter-structure edges.

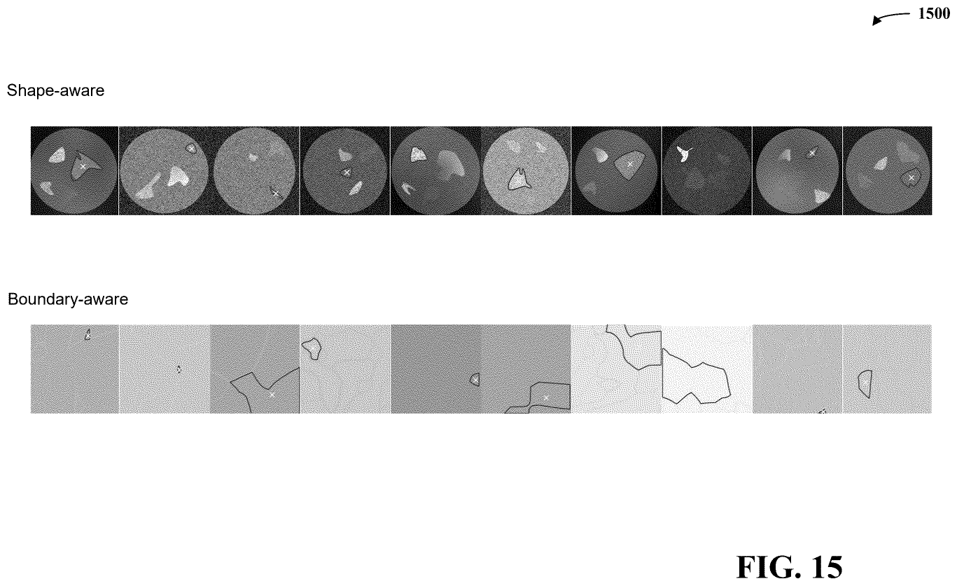

Next, FIG. 15 depicts examples of synthetic data generated using a shape-aware module (top) and a boundary-aware module (bottom) in accordance with one or more embodiments described herein. In the top row, the shape-aware module can be used to synthesize a variety of anatomical shapes that mimic real-world complexity. Each synthetic image in this row includes one or more foreground structures embedded in noisy or textured backgrounds, with shapes generated using Bézier curves or similar parametric models. These shapes can vary widely in size, orientation, and curvature, offering diverse examples for model training. Each instance includes a segmentation prompt, marked by a yellow “X,” and a red contour illustrating the corresponding mask or segmentation boundary. This setup can enable a segmentation model to learn robust representations for irregular and variable anatomical forms under realistic imaging conditions. In the bottom row, the boundary-aware module can be employed to generate synthetic data that features tightly clustered anatomical structures with realistic shared boundaries. These examples emphasize boundary contrast and structural adjacency, simulating the challenges often encountered in clinical scenarios where neighboring tissues or organs are difficult to separate. The red contours in this row outline the synthetic boundaries between adjacent structures, and yellow prompt markers again indicate the input guidance. These images can be created by generating multiclass label maps followed by controlled erosion operations to form thin, ambiguous boundaries between classes. The combination of boundary ambiguity and subtle contrast can train a model to be more precise in distinguishing and delincating abutting anatomical regions. This figure highlights how both modules can be used to generate targeted, task-specific synthetic datasets that improve model generalization across different segmentation scenarios.

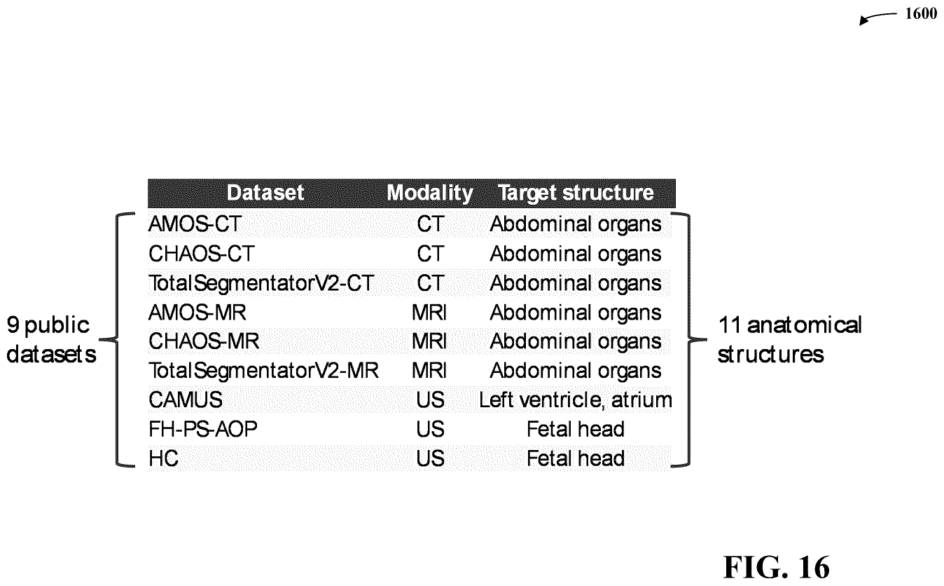

Next, FIG. 16 illustrates a summary of evaluation datasets used to validate performance across diverse clinical imaging scenarios in accordance with one or more embodiments described herein. The figure presents a tabulated breakdown of nine publicly available medical image datasets, listed under the “Dataset” column. Each dataset is associated with a specific imaging modality, including computed tomography (CT), magnetic resonance imaging (MRI), and ultrasound (US), as indicated in the “Modality” column. The “Target structure” column identifies the anatomical regions or features to be segmented, such as abdominal organs, the fetal head, or cardiac structures including the left ventricle and atrium. The vertical annotation on the left indicates that these datasets can collectively represent nine independent sources of real-world medical imaging data. On the right, a second annotation highlights that the datasets span segmentation tasks involving 11 distinct anatomical structures. This figure reinforces that the proposed system can be evaluated across a broad range of imaging types and organ-specific tasks, facilitating assessment of generalization performance. The diversity of modalities and anatomical targets also demonstrates the utility of a synthetic pre-training pipeline that can enable consistent performance across heterogeneous clinical domains.

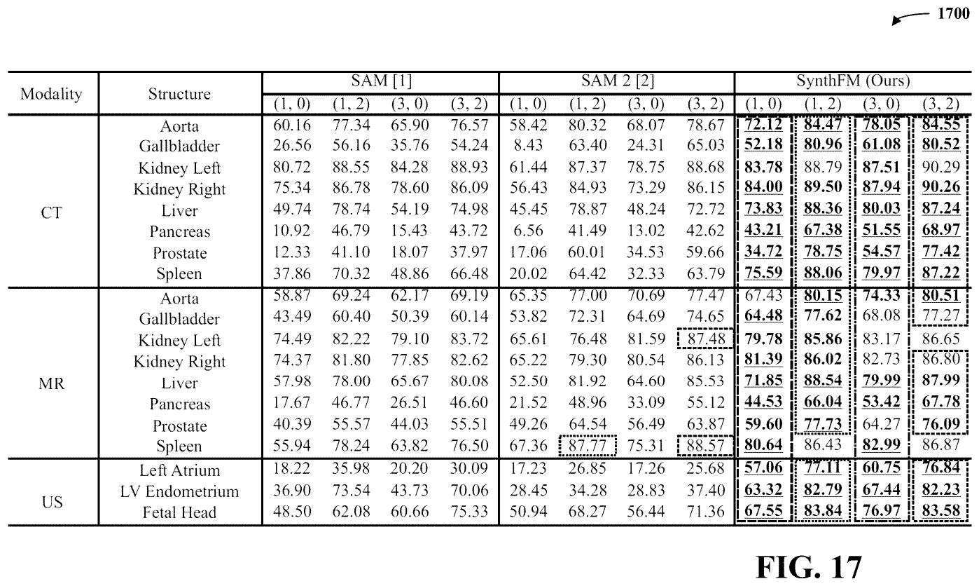

Next, FIG. 17 illustrates a quantitative comparison of segmentation performance across multiple anatomical structures, imaging modalities, and model configurations in accordance with one or more embodiments described herein. The table is organized into three main columns representing model variants: SAM [1], SAM 2 [2], and SynthFM (Ours), each evaluated across four distinct prompt configurations: (1,0), (1,2), (3,0), and (3,2). These prompt pairs correspond to different combinations of positive and negative input prompts provided during segmentation inference. The rows of the table are divided by imaging modality-CT, MR, and US—with each modality including specific anatomical targets such as the aorta, gallbladder, liver, and fetal head. The values in the table reflect segmentation accuracy or similarity metrics, with higher values indicating improved performance. SynthFM (Ours) consistently outperforms both SAM [1] and SAM 2 [2] across nearly all structures and prompt types, demonstrating the effectiveness of pre-training using synthetic data generated by the disclosed framework. This figure supports the broader claim that synthetic pre-training using anatomically realistic and diverse datasets can lead to improved generalization and segmentation fidelity, even across heterogeneous tasks and modalities. By incorporating multiple prompt strategies, the evaluation highlights the robustness of SynthFM to different prompt configurations while maintaining high accuracy across a broad spectrum of clinical segmentation tasks.

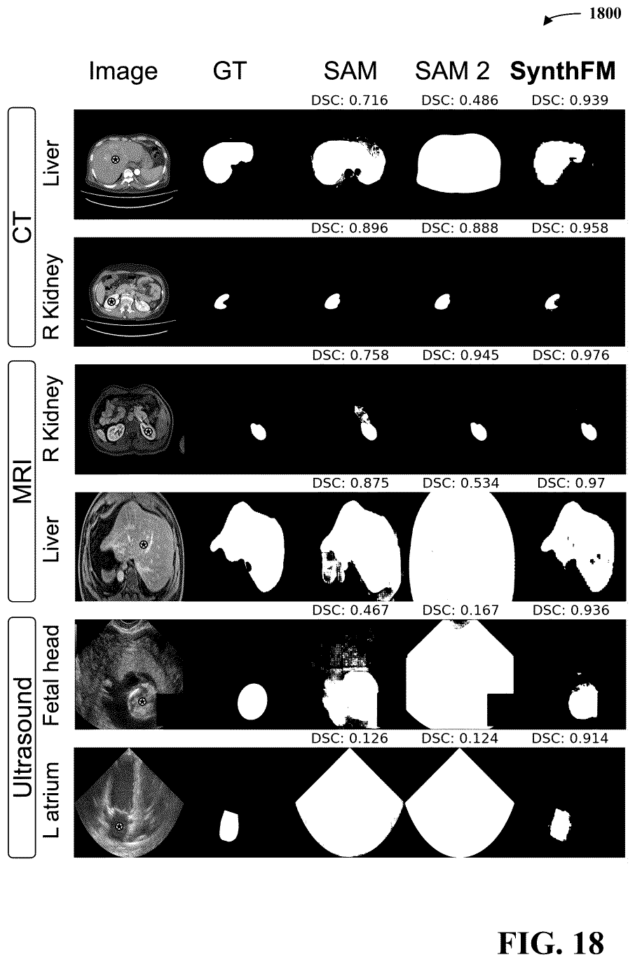

Next, FIG. 18 illustrates a visual comparison of segmentation performance across three models-SAM, SAM 2, and SynthFM-using representative clinical imaging samples for different anatomical structures and modalities, in accordance with one or more embodiments described herein. Each row of the figure corresponds to a specific anatomical region imaged with a distinct modality: CT (right kidney and liver), MRI (right kidney and liver), and ultrasound (fetal head and left atrium). The columns compare ground truth (GT) masks with segmentation outputs produced by the respective models. The ground truth (GT) column provides expert-labeled segmentations used as a reference for qualitative evaluation. The subsequent columns visualize the predicted segmentation overlays from SAM, SAM 2, and SynthFM models, respectively, each accompanied by a corresponding Dice Similarity Coefficient (DSC) score. Higher DSC values indicate better overlap with the ground truth and, thus, higher segmentation accuracy. As shown, SynthFM achieves the most consistent and accurate segmentation across all examples. The masks generated by SynthFM more closely align with the anatomical boundaries, while baseline models exhibit significant inaccuracies and undersegmentation or oversegmentation. The results validate that synthetic pre-training using the SynthFM framework can enhance generalizability across modalities and anatomical targets, yielding reliable performance even in challenging imaging scenarios.

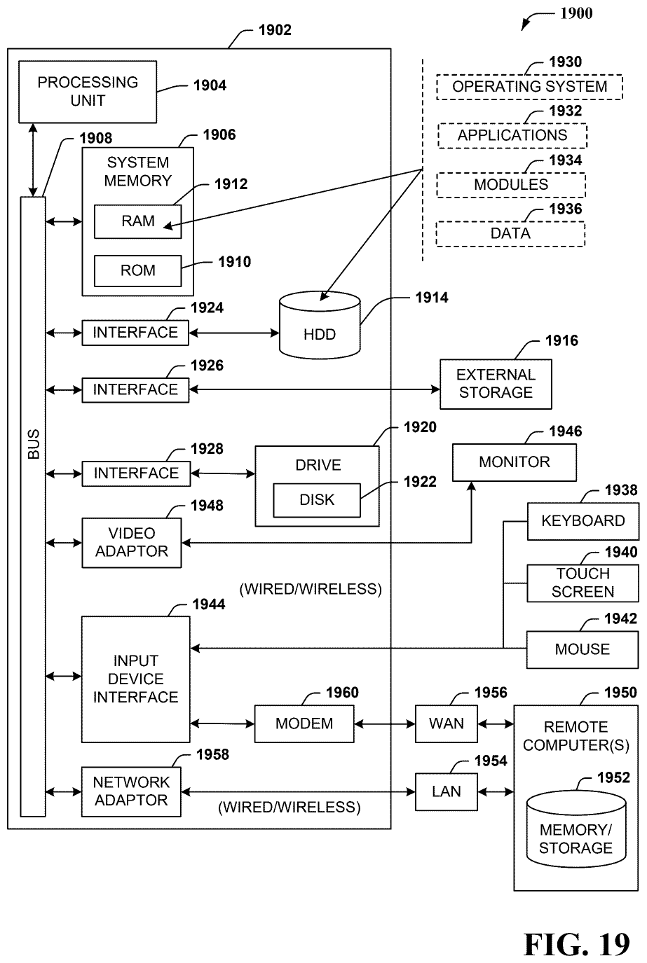

In order to provide additional context for various embodiments described herein, FIG. 19 and the following discussion are intended to provide a brief, general description of a suitable computing environment 1900 in which the various embodiments of the embodiment described herein can be implemented. While the embodiments have been described above in the general context of computer-executable instructions that can run on one or more computers, those skilled in the art will recognize that the embodiments can also be implemented in combination with other program modules or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multi-processor computer systems, minicomputers, mainframe computers, Internet of Things (IOT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can also be practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 19, the example environment 1900 for implementing various embodiments of the aspects described herein includes a computer 1902, the computer 1902 including a processing unit 1904, a system memory 1906 and a system bus 1908. The system bus 1908 couples system components including, but not limited to, the system memory 1906 to the processing unit 1904. The processing unit 1904 can be any of various commercially available processors. Dual microprocessors and other multi-processor architectures can also be employed as the processing unit 1904.

The system bus 1908 can be any of several types of bus structure that can further interconnect to a memory bus (with or without a memory controller), a peripheral bus, and a local bus using any of a variety of commercially available bus architectures. The system memory 1906 includes ROM 1910 and RAM 1912. A basic input/output system (BIOS) can be stored in a non-volatile memory such as ROM, erasable programmable read only memory (EPROM), EEPROM, which BIOS contains the basic routines that help to transfer information between elements within the computer 1902, such as during startup. The RAM 1912 can also include a high-speed RAM such as static RAM for caching data.

The computer 1902 further includes an internal hard disk drive (HDD) 1914 (e.g., EIDE, SATA), one or more external storage devices 1916 (e.g., a magnetic floppy disk drive (FDD) 1916, a memory stick or flash drive reader, a memory card reader, etc.) and a drive 1920, e.g., such as a solid state drive, an optical disk drive, which can read or write from a disk 1922, such as a CD-ROM disc, a DVD, a BD, etc. Alternatively, where a solid state drive is involved, disk 1922 would not be included, unless separate. While the internal HDD 1914 is illustrated as located within the computer 1902, the internal HDD 1914 can also be configured for external use in a suitable chassis (not shown). Additionally, while not shown in environment 1900, a solid state drive (SSD) could be used in addition to, or in place of, an HDD 1914. The HDD 1914, external storage device(s) 1916 and drive 1920 can be connected to the system bus 1908 by an HDD interface 1924, an external storage interface 1926 and a drive interface 1928, respectively. The interface 1924 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1902, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1912, including an operating system 1930, one or more application programs 1932, other program modules 1934 and program data 1936. All or portions of the operating system, applications, modules, or data can also be cached in the RAM 1912. The systems and methods described herein can be implemented utilizing various commercially available operating systems or combinations of operating systems.

Computer 1902 can optionally comprise emulation technologies. For example, a hypervisor (not shown) or other intermediary can emulate a hardware environment for operating system 1930, and the emulated hardware can optionally be different from the hardware illustrated in FIG. 19. In such an embodiment, operating system 1930 can comprise one virtual machine (VM) of multiple VMs hosted at computer 1902. Furthermore, operating system 1930 can provide runtime environments, such as the Java runtime environment or the .NET framework, for applications 1932. Runtime environments are consistent execution environments that allow applications 1932 to run on any operating system that includes the runtime environment. Similarly, operating system 1930 can support containers, and applications 1932 can be in the form of containers, which are lightweight, standalone, executable packages of software that include, e.g., code, runtime, system tools, system libraries and settings for an application.

Further, computer 1902 can be enabled with a security module, such as a trusted processing module (TPM). For instance, with a TPM, boot components hash next in time boot components, and wait for a match of results to secured values, before loading a next boot component. This process can take place at any layer in the code execution stack of computer 1902, e.g., applied at the application execution level or at the operating system (OS) kernel level, thereby enabling security at any level of code execution.

A user can enter commands and information into the computer 1902 through one or more wired/wireless input devices, e.g., a keyboard 1938, a touch screen 1940, and a pointing device, such as a mouse 1942. Other input devices (not shown) can include a microphone, an infrared (IR) remote control, a radio frequency (RF) remote control, or other remote control, a joystick, a virtual reality controller or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1904 through an input device interface 1944 that can be coupled to the system bus 1908, but can be connected by other interfaces, such as a parallel port, an IEEE 1394 serial port, a game port, a USB port, an IR interface, a BLUETOOTH® interface, etc.

A monitor 1946 or other type of display device can also be connected to the system bus 1908 via an interface, such as a video adapter 1948. In addition to the monitor 1946, a computer typically includes other peripheral output devices (not shown), such as speakers, printers, etc.

The computer 1902 can operate in a networked environment using logical connections via wired or wireless communications to one or more remote computers, such as a remote computer(s) 1950. The remote computer(s) 1950 can be a workstation, a server computer, a router, a personal computer, portable computer, microprocessor-based entertainment appliance, a peer device or other common network node, and typically includes many or all of the elements described relative to the computer 1902, although, for purposes of brevity, only a memory/storage device 1952 is illustrated. The logical connections depicted include wired/wireless connectivity to a local area network (LAN) 1954 or larger networks, e.g., a wide area network (WAN) 1956. Such LAN and WAN networking environments are commonplace in offices and companies, and facilitate enterprise-wide computer networks, such as intranets, all of which can connect to a global communications network, e.g., the Internet.

When used in a LAN networking environment, the computer 1902 can be connected to the local network 1954 through a wired or wireless communication network interface or adapter 1958. The adapter 1958 can facilitate wired or wireless communication to the LAN 1954, which can also include a wireless access point (AP) disposed thereon for communicating with the adapter 1958 in a wireless mode.

When used in a WAN networking environment, the computer 1902 can include a modem 1960 or can be connected to a communications server on the WAN 1956 via other means for establishing communications over the WAN 1956, such as by way of the Internet. The modem 1960, which can be internal or external and a wired or wireless device, can be connected to the system bus 1908 via the input device interface 1944. In a networked environment, program modules depicted relative to the computer 1902 or portions thereof, can be stored in the remote memory/storage device 1952. It will be appreciated that the network connections shown are example and other means of establishing a communications link between the computers can be used.

When used in either a LAN or WAN networking environment, the computer 1902 can access cloud storage systems or other network-based storage systems in addition to, or in place of, external storage devices 1916 as described above, such as but not limited to a network virtual machine providing one or more aspects of storage or processing of information. Generally, a connection between the computer 1902 and a cloud storage system can be established over a LAN 1954 or WAN 1956 e.g., by the adapter 1958 or modem 1960, respectively. Upon connecting the computer 1902 to an associated cloud storage system, the external storage interface 1926 can, with the aid of the adapter 1958 or modem 1960, manage storage provided by the cloud storage system as it would other types of external storage. For instance, the external storage interface 1926 can be configured to provide access to cloud storage sources as if those sources were physically connected to the computer 1902.

The computer 1902 can be operable to communicate with any wireless devices or entities operatively disposed in wireless communication, e.g., a printer, scanner, desktop or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

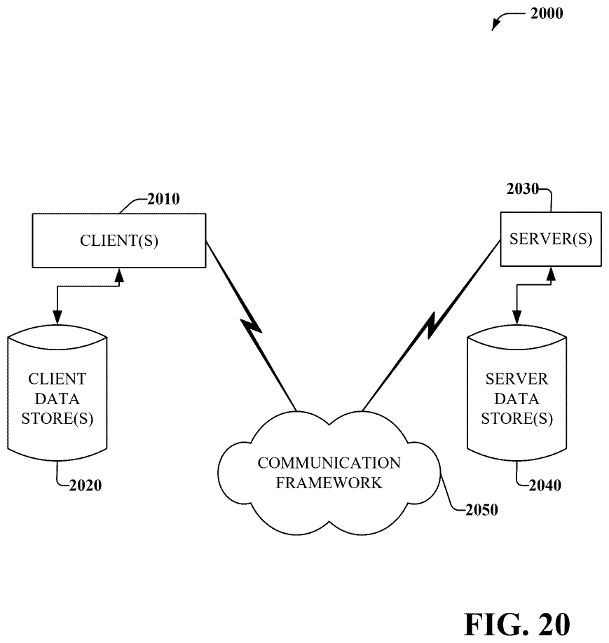

FIG. 20 is a schematic block diagram of a sample computing environment 2000 with which the disclosed subject matter can interact. The sample computing environment 2000 includes one or more client(s) 2010. The client(s) 2010 can be hardware or software (e.g., threads, processes, computing devices). The sample computing environment 2000 also includes one or more server(s) 2030. The server(s) 2030 can also be hardware or software (e.g., threads, processes, computing devices). The servers 2030 can house threads to perform transformations by employing one or more embodiments as described herein, for example. One possible communication between a client 2010 and a server 2030 can be in the form of a data packet adapted to be transmitted between two or more computer processes. The sample computing environment 2000 includes a communication framework 2050 that can be employed to facilitate communications between the client(s) 2010 and the server(s) 2030. The client(s) 2010 are operably connected to one or more client data store(s) 2020 that can be employed to store information local to the client(s) 2010. Similarly, the server(s) 2030 are operably connected to one or more server data store(s) 2040 that can be employed to store information local to the servers 1130.

Various embodiments can be a system, a method, an apparatus or a computer program product at any possible technical detail level of integration. The computer program product can include a computer readable storage medium (or media) having computer readable program instructions thereon for causing a processor to carry out aspects of various embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, fire walls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of various embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform various aspects.

Various aspects are described herein with reference to flowchart illustrations or block diagrams of methods, apparatus (systems), and computer program products according to various embodiments. It will be understood that each block of the flowchart illustrations or block diagrams, and combinations of blocks in the flowchart illustrations or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowcharts and block diagrams in the Figures illustrate the architecture, functionality, and operation of possible implementations of systems, methods, and computer program products according to various embodiments. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that various aspects can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can execute from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. As used herein, the term “and/or” is intended to have the same meaning as “or.” Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

The disclosure herein describes non-limiting examples. For ease of description or explanation, various portions of the herein disclosure utilize the term “each,” “every,” or “all” when discussing various examples. Such usages of the term “each,” “every,” or “all” are non-limiting. In other words, when the herein disclosure provides a description that is applied to “each,” “every,” or “all” of some particular object or component, it should be understood that this is a non-limiting example, and it should be further understood that, in various other examples, it can be the case that such description applies to fewer than “each,” “every,” or “all” of that particular object or component.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various non-limiting aspects of various embodiments described herein are presented in the following clauses.
Clause 1: A system, comprising: a processor that executes computer-executable components stored in a non-transitory computer-readable memory, wherein the computer-executable components comprise: a synthetic data generation component that generates biologically-inspired synthetic data that approximates a task-specific data manifold of a medical image from a radiomic features perspective; an artificial intelligence component that uses an artificial intelligence model to learn relevant representations of the synthetic data for an at least one image task; and a training component that utilizes the relevant representations and the artificial intelligence model to generate a task-specific model for the at least one image analysis task.
Clause 2: The system of any preceding clause, further comprising a shape aware synthetic tool component that employs Bézier curve-based object generation to generate a diverse set of synthetic shapes and structures to train the artificial intelligence model.
Clause 3: The system of any preceding clause, further comprising a contrast and texture tool component that employs noise models, image morphological and intensity operations, and generative AI methods to generate a diverse set of contrasts or textures to train the artificial intelligence model.
Clause 4: The system of any preceding clause, further comprising a boundary-aware synthetic tool component that generates random structures that share boundaries to train the artificial intelligence model.
Clause 5: The system of any preceding clause, wherein the synthetic data generation component generates synthetic medical images emulating contrast, noise, and texture characteristics of real medical images.
Clause 6: The system of any preceding clause, wherein the shape aware synthetic tool component varies the number of Bézier curve control points to generate anatomical structures of differing shape complexity.
Clause 7: The system of any preceding clause, wherein the contrast and texture tool component includes a noise library configured to apply at least one of: Poisson noise, Rician noise, speckle noise, or Perlin noise to the synthetic data.
Clause 8: The system of any preceding clause, wherein the boundary-aware synthetic tool component comprises an erosion module that applies a randomly determined number of erosion operations to clusters within a label map to create narrow boundaries between adjacent synthetic structures.
Clause 9: The system of any preceding clause, wherein the synthetic data generation component modulates contrast by assigning intensity values to foreground and background regions of a label map using randomized intensity variations.
Clause 10: A computer-implemented method that utilizes a processor that executes computer executable components stored in memory to perform the following acts: generating synthetic data that approximates a task-specific data manifold of a medical image from a radiomic features perspective; using an artificial intelligence model to learn relevant representations of the synthetic data to generate task-specific outputs for an at least one image task; and utilizing the relevant representations and the artificial intelligence model to generate a task-specific model for the at least one image analysis task.
Clause 11: The method of any preceding clause, further comprising: employing Bézier curve-based object generation to generate a diverse set of synthetic shapes and structures to train the task-specific model.
Clause 12: The method of any preceding clause, further comprising: applying noise models, image morphological and intensity operations, and generative AI methods to generate a diverse set of contrasts or textures to train the task-specific model.
Clause 13: The method of any preceding clause, further comprising: generating random structures that share boundaries to train the task-specific model.
Clause 14: The method of any preceding clause, wherein the Bézier curve-based object generation comprises randomly selecting a number of control points for each shape to increase anatomical variability.
Clause 15: The method of any preceding clause, wherein generating random structures that share boundaries comprises: generating a multiclass label map comprising multiple clusters; selecting a subset of the clusters; and performing a randomly selected number of erosion operations on the selected clusters to define thin boundaries.
Clause 16: The method of any preceding clause, further comprising modulating the contrast between structures and background by assigning intensity values to foreground and background regions of a label map using randomized intensity variations based on task-specific parameters.
Clause 17: The method of any preceding clause, further comprising generating synthetic training images on-the-fly during model training without pre-generating a fixed dataset.
Clause 18: The method of any preceding clause, further comprising saving the synthetic images and metadata specifying generation parameters to enable reproducibility, dataset verification, and offline reuse.
Clause 19: The method of any preceding clause, wherein the artificial intelligence model is a general-purpose model, and wherein the method further comprises training the general-purpose model using the synthetic data to produce the task-specific model.
Clause 20: A computer program product for facilitating training of an image segmentation model, the computer program product comprising a non-transitory computer-readable memory having program instructions embodied therewith, the program instructions executable by a processor to cause the processor to: generate biologically-inspired synthetic data that closely approximates a task-specific data manifold of a medical image from a radiomic features perspective; use an artificial intelligence model to encode features of the synthetic data for at least one image analysis task; and utilize the features of the synthetic data and the artificial intelligence model to generate a task-specific model for the at least one image analysis task.

In various cases, any suitable combination or combinations of clauses 1-20 can be implemented.

What is claimed is:

1. A system, comprising:
a processor that executes computer-executable components stored in a non-transitory computer-readable memory, wherein the computer-executable components comprise:
a synthetic data generation component that generates biologically-inspired synthetic data that approximates a task-specific data manifold of a medical image from a radiomic features perspective;
an artificial intelligence component that uses an artificial intelligence model to learn relevant representations of the synthetic data for an at least one image task; and
a training component that utilizes the relevant representations and the artificial intelligence model to generate a task-specific model for the at least one image analysis task.

2. The system of claim 1, further comprising a shape aware synthetic tool component that employs Bézier curve-based object generation to generate a diverse set of synthetic shapes and structures to train the artificial intelligence model.

3. The system of claim 1, further comprising a contrast and texture tool component that employs noise models, image morphological and intensity operations, and generative AI methods to generate a diverse set of contrasts or textures to train the artificial intelligence model.

4. The system of claim 1, further comprising a boundary-aware synthetic tool component that generates random structures that share boundaries to train the artificial intelligence model.

5. The system of claim 1, wherein the synthetic data generation component generates synthetic medical images emulating contrast, noise, and texture characteristics of real medical images.

6. The system of claim 1, wherein the shape aware synthetic tool component varies the number of Bézier curve control points to generate anatomical structures of differing shape complexity.

7. The system of claim 3, wherein the contrast and texture tool component includes a noise library configured to apply at least one of: Poisson noise, Rician noise, speckle noise, or Perlin noise to the synthetic data.

8. The system of claim 1, wherein the boundary-aware synthetic tool component comprises an erosion module that applies a randomly determined number of erosion operations to clusters within a label map to create narrow boundaries between adjacent synthetic structures.

9. The system of claim 1, wherein the synthetic data generation component modulates contrast by assigning intensity values to foreground and background regions of a label map using randomized intensity variations.

10. A computer-implemented method, comprising:
generating synthetic data that approximates a task-specific data manifold of a medical image from a radiomic features perspective;
using an artificial intelligence model to learn relevant representations of the synthetic data for an at least one image task; and
utilizing the relevant representations and the artificial intelligence model to generate a task-specific model for the at least one image analysis task.

11. The computer-implemented method of claim 10, further comprising: employing Bézier curve-based object generation to generate a diverse set of synthetic shapes and structures to train the task-specific model.

12. The computer-implemented method of claim 10, further comprising: applying noise models, image morphological and intensity operations, and generative AI methods to generate a diverse set of contrasts or textures to train the task-specific model.

13. The computer-implemented method of claim 10, further comprising: generating random structures that share boundaries to train the task-specific model.

14. The computer-implemented method of claim 11, wherein the Bézier curve-based object generation comprises randomly selecting a number of control points for each shape to increase anatomical variability.

15. The computer-implemented method of claim 13, wherein generating random structures that share boundaries comprises:
generating a multiclass label map comprising multiple clusters;
selecting a subset of the clusters; and
performing a randomly selected number of erosion operations on the selected clusters to define thin boundaries.

16. The computer-implemented method of claim 10, further comprising modulating the contrast between structures and background by assigning intensity values to foreground and background regions of a label map using randomized intensity variations based on task-specific parameters.

17. The computer-implemented method of claim 10, further comprising generating synthetic training images on-the-fly during model training without pre-generating a fixed dataset.

18. The computer-implemented method of claim 10, further comprising saving the synthetic images and metadata specifying generation parameters to enable reproducibility, dataset verification, and offline reuse.

19. The computer-implemented method of claim 10, wherein the artificial intelligence model is a general-purpose model, and wherein the method further comprises training the general-purpose model using the synthetic data to produce the task-specific model.

20. A computer program product for facilitating training of an image segmentation model, the computer program product comprising a non-transitory computer-readable memory having program instructions embodied therewith, the program instructions executable by a processor to cause the processor to:
generate biologically-inspired synthetic data that approximates a task-specific data manifold of a medical image from a radiomic features perspective;
use an artificial intelligence model to encode features of the synthetic data for at least one image analysis task; and
utilize the features of the synthetic data and the artificial intelligence model to generate a task-specific model for the at least one image analysis task.
